(19) United States
(12) Patent Application Publication
(10) Pub. No.: US 2025/0375435 A1
(43) Pub. Date: Dec. 11, 2025

(54) COMPOUNDS FOR TREATING INFECTIONS

(71) Applicant: Ohio State Innovation Foundation, Columbus, OH (US)

(72) Inventors: Brian AHMER, Lewis Center, OH (US); Venkat GOPALAN, Columbus, OH (US); Anice SABAG-DAIGLE, Columbus, OH (US); Erin BOULANGER, Columbus, OH (US); Jamison LAW, Columbus, OH (US); Sai Sravya Varali KOVVALI, Columbus, OH (US); Anindita SENGUPTA, Nottingham (GB); Pankajavalli THIRUGNANASAMBANTHAM, Lewis Center, OH (US); Steffen LINDERT, Columbus, OH (US); Austin COOL, Whitestown, IN (US)

(21) Appl. No.: 18/877,007

(22) PCT Filed: Jun. 21, 2023
(86) PCT No.: PCT/US2023/025869

§ 371 Date: Dec. 19, 2024

Related U.S. Application Data
(60) Provisional application No. 63/354,038, filed on Jun. 21, 2022.
(60) Provisional application No. 63/354,805, filed on Jun. 23, 2022.
(60) Provisional application No. 63/392,200, filed on Jul. 26, 2022.

(51) Int. Cl. A61K 31/454; A61K 9/00; A61P 31/04

(52) U.S. Cl. CPC A61K 31/454; A61K 9/0019; A61P 31/04

(57) ABSTRACT

Disclosed herein are compositions and methods for treating a Salmonella infection. Disclosed herein are compositions and methods for inhibiting FraB in Salmonella. Disclosed herein are methods of increasing 6-phospho-fructose-aspartate within Salmonella bacteria.

### CROSS-REFERENCE TO RELATED APPLICATION

This application claims the benefit of U.S. Provisional Applications 63/354,038, filed Jun. 21, 2022, 63/354,805, filed Jun. 23, 2022, and 63/392,200, filed Jul. 26, 2022, the content of which are hereby incorporated in their entirety.

### STATEMENT OF GOVERNMENT SUPPORT

This invention was made with government support under grant/contract number R01 AI116119 and R01 AI140541 awarded by the National Institutes of Health. The government has certain rights in the invention.

### BACKGROUND

Salmonella enterica is among the world's most significant causes of morbidity and mortality due to food-borne disease. A few serovars of Salmonella cause typhoid fever, while hundreds of non-typhoidal serovars can cause an acute gastroenteritis that is characterized by an inflammatory diarrhea and fever. There are no vaccines approved for human use that protect against the non-typhoidal serovars. There are also no narrow-spectrum antibiotics that can target Salmonella within the gastrointestinal tract to reduce the duration and severity of infection.

Salmonella enterica can utilize a nutrient called fructose-asparagine (F-Asn) using a catabolic pathway that is rare among Gram-negative bacteria, and not common among Gram-positive bacteria either. One enzyme within this pathway, FraB, is a potential drug target largely specific for Salmonella. When FraB is inhibited (or mutated), utilization of F-Asn results in the accumulation of a toxic product within the cell, 6-phospho-fructose-aspartate (6-P-F-Asp).

There remains a need for improved compositions and methods for treating Salmonella infection. There remains a need for improved compositions and methods for inhibiting FraB. There remains a need for improved compositions and methods for providing increased levels of 6-phospho-fructose-aspartate within Salmonella bacteria.

### BRIEF DESCRIPTION OF THE FIGURES

FIGS. 1-8 each depict dose response curves for compounds that can inhibit the growth of Salmonella. Each of the seven compounds, and the positive control compound K19, were tested against four bacterial strains at seven concentrations to obtain dose-response curves. The IC₅₀ for each compound on each of the four bacterial strains is shown to the right of each graph. ASD200 and ASD201 are wild-type at the tolC locus, while EFB077 and EFB078 are mutated at the tolC locus. Each of the compounds appears to be affected by efflux as the IC₅₀s are one to three logs lower in the tolC mutant background. All the compounds appear partially fra-dependent as the IC₅₀s are higher in the Δ(fraR-fraBDAE) mutant strains (ASD201 and EFB078) than in their isogenic fra+strains (ASD200 and EFB077).

FIG. 1 depicts IC₅₀ data for the compound having the structure:

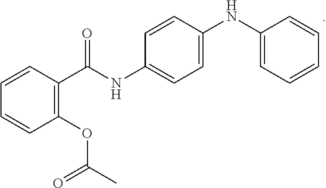 [SMILES: CC(=O)Oc1ccccc1C(=O)Nc1ccc(Nc2ccccc2)cc1]

FIG. 2 depicts IC₅₀ data for the compound having the structure:

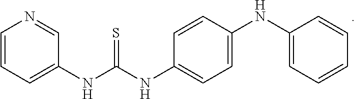 [SMILES: S=C(Nc1ccc(Nc2ccccc2)cc1)Nc1cccnc1]

FIG. 3 depicts IC₅₀ data for the compound having the structure:

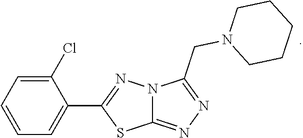 [SMILES: Clc1ccccc1-c1nn2c(CN3CCCCC3)nnc2s1]

FIG. 4 depicts IC₅₀ data for compound the compound having the structure:

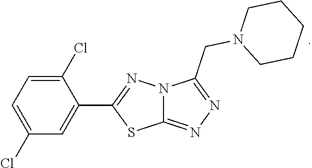 [SMILES: Clc1ccc(Cl)c(-c2nn3c(CN4CCCCC4)nnc3s2)c1]

FIG. 5 depicts IC₅₀ data for the compound having the structure:

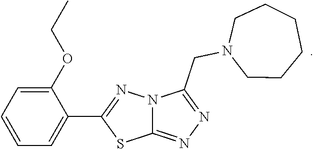 [SMILES: CCOc1ccccc1-c1nn2c(CN3CCCCCC3)nnc2s1]

FIG. 6 depicts IC₅₀ data for compound K19, having the structure:

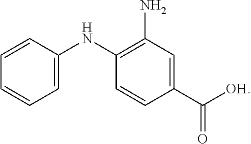 [SMILES: Nc1cc(C(=O)O)ccc1Nc1ccccc1]

FIG. 7 depicts IC₅₀ data for the compound having the structure:

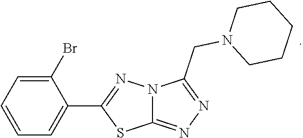 [SMILES: Brc1ccccc1-c1nn2c(CN3CCCCC3)nnc2s1]

FIG. 8 depicts IC₅₀ data for the compound having the structure:

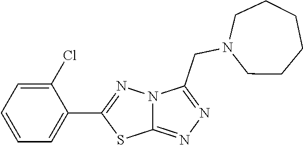 [SMILES: Clc1ccccc1-c1nn2c(CN3CCCCCC3)nnc2s1]

FIG. 9A depicts single infections of streptomycin-treated Swiss Webster mice and then harvested on day 4 post-infection for each strain. The wild-type strain is JLD1214 which is 14028 marked with a neutral chloramphenicol resistance cassette. The fraB E214A point mutant strain is ASD1312. Mock samples were mice that were not inoculated with Salmonella and strep only mice were mice that received only streptomycin-treatment and allowed to recover.

FIG. 9B depicts a biological replicate of the two shown in FIG. 9A and depicts F-Asn concentration determined by LC-MS/MS in feces collected on day 4 post-infection.

FIG. 9C depicts a biological replicate of the two shown in FIG. 9A and depicts histopathology scores for inflammation in tissue samples obtained from the proximal colon day 4 post-infection.

FIG. 9D depicts a biological replicate of the two shown in FIG. 9A and depicts the abundance of Enterobacteriaceae or non-Enterobacteriaceae that encode fraBD based on 16s rRNA.

FIG. 9E depicts results from two separate experiments (5 mice per group in each experiment, 10 mice per group total). The inoculum was 1.2×10⁸ and 5×10⁷ for the wild-type and 1.5×10⁸ and 2.8×10⁷ for the fraB E214A mutant. When probiotic is present, CFU of each strain was determined by plating on media selective for WT (LB cam) and the mutant (LB cam). The probiotic strain was plated on LBkan and is graphed on the right y-axis. Probiotic counts are indicated by the blue squares. Some groups were administered 735 mg of F-Asn intragastrically (i.g.) daily as indicated on the x-axis. There is no statistical difference between any of the probiotic groups.

FIG. 9F depicts a biological replicate of the two shown in FIG. 9A with the addition of probiotic and depicts F-Asn concentration determined by LC-MS/MS in feces collected on day 4 post-infection.

FIG. 9G depicts a biological replicate of the two shown in FIG. 9A with the addition of probiotic and depicts histopathology scores for inflammation in tissue samples obtained from the proximal colon day 4 post-infection.

FIG. 9H depicts a biological replicate of the two shown in FIG. 9A with the addition of probiotic and depicts the abundance of Enterobacteriaceae or non-Enterobacteriaceae that encode fraBD based on 16s rRNA.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E.L. Stereochemistry of Carbon Compounds, McGraw-Hill, NY, 1962; and Wilen, S.H., Tables of Resolving Agents and Optical Resolutions p. 268, E.L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups. In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂—). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1or more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₁₆ alkyl”). In some embodiments, a heteroalkyl group is a saturated group having 1 to14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1to 10 carbon atoms and/or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1carbon atom and 1heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C3), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like Additional examples of alkenyl include heptenyl (C₇), octenyl (C₈), octatrienyl (C₈), and the like Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

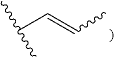

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl. The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”).

The one or more carbon-carbon triple bonds can be internal (such as in 2-butynyl) or terminal (such as in 1-butynyl). Examples of C_{2 4} alkynyl groups include, without limitation, ethynyl (C₂), 1-propynyl (C₃), 2-propynyl (C₃), 1-butynyl (C₄), 2-butynyl (C₄), and the like. Examples of C₂₆alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and lor more heteroatoms within the parent chain (“heteroC₂₋₉ alkynyl”). In some embodiments, a heteroalkynyl group has 2 to 8 carbon atoms, at least one triple bond, and lor more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈ carbocyclyl groups include, without limitation, the aforementioned C₃₋₆carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo [2.2.1] heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur. Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5 -membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃ ⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})2, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(—O)(OR^{cc})₂, —OP(═O)(R^{aa})₂, —OP(═O)(O_{R})₂, —P(═O)(N(R^{bb})₂)_{2,} —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc}C)₂, —P(RC)₃ ⁺X⁻, —P(OR^{cc})₃ ⁺X⁻, —P(R^{cc})₄, —P(OR^{cc})₂, —OP(R^{cc})₂, —OP(R^{cc})₃ ⁺X⁻, —OP(OR^{cc})₂, —OP(OR^{cc})₃ ⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})², —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃ ⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(—NR^{ff})OR^{cc}, —OC(═NR^{ff})R^{ee}, —OC(═NR^{ff})OR^{ee}, —C(═NR^{ff})N(R^{ff})₂, —OC (═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR)₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form —O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON (C₁₋₆ alkyl)₂, —N (C₁₋₆ alkyl)₂, —N(C₁₋₆ alkyl); ⁺X⁻, —NH(C₁₋₆ alkyl)₂ ⁺X⁻, —NH₂(C₁₋₆ alkyl) ⁺X⁻, —NH₃ ⁺X⁻, —N (OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC (═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C_{I-6} alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₄ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form —O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃ ⁺X⁻, —OP(OR)₂, —OP(OR^{cc})₃ ⁺X⁻, —OP(—O)(R^{aa})₂, —OP(═O)(OR^{cc})₂, and —OP(═O)(N(R^{bb})₂)₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted ammino group.

The term “monosubstituted amino” refers to an amino group wherein the nitrogen atom directly attached to the parent molecule is substituted with one hydrogen and one group other than hydrogen, and includes groups selected from —NH(R_{bb}), —NHC(═O)R^{aa}, —NHCO₂R^{aa}, —NHC(—O)N(R_{bb})₂, —NHC(—NR_{bb})N(R_{bb})₂, —NHSO₂R^{aa}, —NHP(—O)(OR^{cc})₂, and —NHP(—O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, 'NR^{bb}C(═O)R^{aa}, —NR^{bb}CO₂R^{aa}, —NR^{bb}C(═O)N(R^{bb})₂, —NR^{bb}C(—NR^{bb})N(R^{bb})₂, —NR^{bb}SO₂R^{aa}, —NR^{bb}P(═O)(OR)₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃ ⁺X⁻, wherein R^{bb} and X⁻ are as defined herein.

The term “sulfonyl” refers to a group selected from —SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein Raa is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(—NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono-or di-aliphaticamino, mono-or di-heteroaliphaticamino, mono-or dialkylamino, mono-or di-heteroalkylamino, mono-or di-arylamino, or mono-or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, butare not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(RC)₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(—O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

As used herein, a chemical bond depicted: [symbol] represents either a single, double, or triple bond, valency permitting. By way of example,

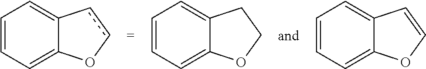

An electron-withdrawing group is a functional group or atom that pulls electron density towards itself, away from other portions of the molecule, e.g., through resonance and/or inductive effects. Exemplary electron-withdrawing groups include F, CI, Br, I, NO₂, CN, SO₂R, SO₃R, SO₂NR₂, C(O)R^{1a}, C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

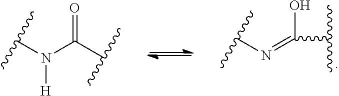

The prevalence of one tautomeric form over another will depend both on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

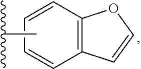

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are compound for the treatment of infections, for example Salmonellainfections.

In certain implementations, the compound is a compound of Formula (1):

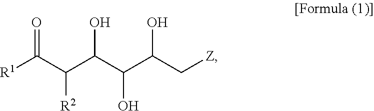

or a pharmaceutically acceptable salt thereof, wherein:

R¹ is OR^{1a} or N(R^{1a})₂, wherein R^{1a} is in each case independently selected from H, C₁₋₈alkyl, aryl, C₁₋₈heteroaryl, C₃₋₈cycloalkyl, or C₁₋₈heterocyclyl;

R² is H or OH;

Z is Z¹—X, wherein Z¹ is null, NH, S, O, CH₂, CH₂O, OCH₂, OCH₂O, CH₂NH, NHCH₂, CH₂S, SCH₂,

X is P(O)(OR^{p})₂, SO₂N(R^{p})₂, SO₂R^{p*}, wherein R^{p} is in each case independently selected from H or C₁₋₈alkyl, and R^{p*} is C₁₋₈alkyl.

In certain implementations the compound of Formula (1) has the structure:

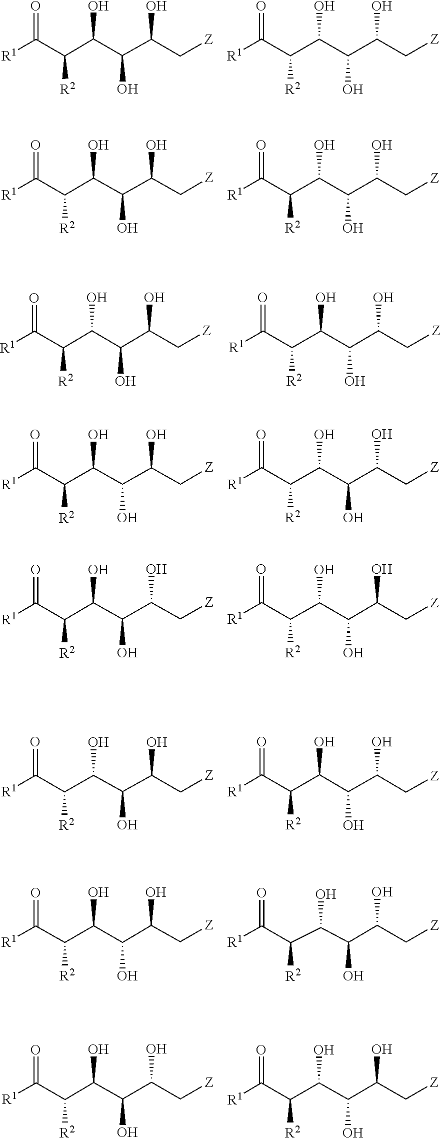

In some implementations Z¹ is null, O, CH₂, CH₂O, OCH₂, or OCH₂O. In some implementations X is P(O)(OH)₂, SO₂NH₂, SO₂CH₃.

In certain implementations, the compound is a compound of Formula (2):

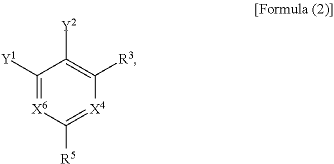

or a pharmaceutically acceptable salt thereof, wherein:

one of Y¹ and Y² is NH₂, and the other is Y*—Ar, wherein Y* is null, NH, or O, and Ar is aryl or C₁₋₈heteroaryl;

X⁴ is N or CR⁴;

X⁶ is N or CR⁶;

R³ is F, Cl, Br, I, NO₂, CN, R^{3*}, OR_{3*}, SR^{3*}, N(R^{3*})₂, SO₃R^{3*}, SO₂R^{3*}, SO₂N(R^{3*})₂, C(O)R^{3*}; C(O)OR^{3*}, OC(O)R^{3*}; C(O)N(R^{3*})₂, N(R^{3*})C(O)R³⁸, OC(O)N(R^{3*})₂, or N(R^{3*})C(O)N(R^{3*})₂, wherein R^{3*} is in each case independently selected from hydrogen, C₁₋₈alkyl, aryl, C₁₋₈heteroaryl, C₃₋₈cycloalkyl, or C₁₋₈heterocyclyl; wherein two or more of R^{3*} may together form a ring, wherein R^{3*} may be substituted one or more times, R²;

R⁴ is F, Cl, Br, I, NO₂, CN, R^{4*}, OR^{4*}, SR^{4*}, N(R^{4*})₂, SO₃R^{4*}, SO₂R^{4*}, SO₂N(R^{4*})₂, C(O)R^{4*}, C(O)OR^{4*}, OC(O)R^{4*}; C(O)N(R^{4*})₂, N(R^{4*})C(O)R^{4*}, OC(O)N(R^{4*})₂, or N(R^{4*})C(O)N(R^{4*})₂, wherein R^{4*} is in each case independently selected from hydrogen, C₁₋₈alkyl, aryl, C₁₋₈heteroaryl, C₃₋₈cycloalkyl, or C₁₋₈heterocyclyl; wherein two or more of R^{4*} may together form a ring, wherein R^{4*} may be substituted one or more times, R₂;

R⁵ is F, Cl, Br, I, NO₂, CN, R^{5*}, OR^{5*}, SR^{5*}, N(R^{5*})₂, SO₃R^{5*}, SO₂R^{5*}, SO₂N(R^{5*})₂, C(O)R^{5*}; C(O)OR^{5*}, OC(O)R^{5*}, C(O)N(R^{5*})₂, N(R^{5*})C(O)R^{5*}, OC(O)N(R^{5*})₂, or N(R^{5*})C(O)N(R^{5*})₂, wherein R^{5*} is in each case independently selected from hydrogen, C₁₋₈alkyl, aryl, C₁₋₈heteroaryl, C₃₋₈cycloalkyl, or C₁₋₈heterocyclyl; wherein two or more of R^{5*} may together form a ring, wherein R^{5*} may be substituted one or more times, R²;

R⁶ is F, Cl, Br, I, NO₂, CN, R^{6*}, OR^{6*}, SR^{6*}, N(R^{6*})₂, SO₃R^{6*}, SO₂R^{6*}, SO₂N(R^{6*})₂, C(O)R^{6 *}; C(O)OR^{6*}, OC(O)R^{6*}; C(O)N(R^{6*})₂, N(R^{6*})C(O)R^{6*}, OC(O)N(R^{6*})₂, or N(R^{6a*})C(O)N(R^{6a*})₂, wherein R^{6*} is in each case independently selected from hydrogen, C₁₋₈alkyl, aryl, C₁₋₈heteroaryl, C₃₋₈cycloalkyl, or C₁₋₈heterocyclyl; wherein two or more of R^{6*} may together form a ring, wherein R^{6*} may be substituted one or more times, R²;

R^{z} is in each case independently selected from F, Cl, Br, I, C₁₋₄alkyl, aryl, heteroaryl, OH, OC₁₋₄alkyl, NH₂, NHC₁₋₄alkyl, or N(C₁₋₄alkyl)₂.

In some implementations Ar is C₆aryl. In certain implementations X⁵ is N or CH. In further implementations Ar is C₆aryl and X⁵ is N or CH.

In certain implementations, Y² is NH₂, and X⁴is C—R⁴, wherein R⁴ is F, Cl, Br, I, CN, R^{4*}, OR^{4*}, C(O)OR^{4*}, or C(O)N(R^{4*})₂. In certain implementations Y² is NH₂, and X⁴ is C—R⁴, wherein R⁴C(O)N(R^{4*})₂, and R^{4*} is independently selected from H, C₁₋₄alkyl, or C₁₋₈heterocyclyl. In some implementations Y² is NH₂, and X⁴ is C—R⁴, wherein R⁴ is C(O)N(R^{4*})₂, wherein one of R^{4*} is H and the other is C₁₋₄alkyl or C₁₋₈heterocyclyl. In other implementations Y² is NH₂, and X⁴ is C—R⁴, wherein R⁴ is C(O)N(R^{4*})₂, wherein both of R^{4*} together form a ring.

In some implementations, the compound of Formula (2) is a compound of Formula (2a):

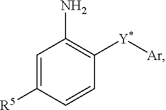

wherein

Y* is NH or O, preferably NH;

Ar is C₆aryl C₁₋₈heteroaryl, C₃₋₈cycloalkyl, or C₁₋₈heterocyclyl; and

R⁵ is C(O)OR^{5*} or C(O)N(R^{5*})₂, preferably C(O)N(R^{5*})₂, wherein R^{5*} is independently selected from H, C₁₋₈alkyl, and C₃₋₈cycloalkyl. In certain implementations R^{5a} is C₁₋₈alkyl substituted by aryl. In certain implementations, R^{5*} is in each case H.

In certain implementations, the compound of Formula (2a) has the formula:

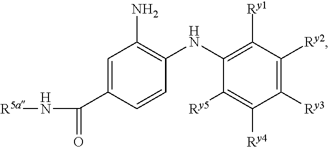

wherein

R^{5a*} is selected from H C₁₋₈alkyl, and C₃₋₈cycloalkyl;

R^{y1} is selected from H, F, Cl, Br, C₁₋₄alkyl, OH, OC₁₋₄alkyl, NH₂, NHC₁₋₄alkyl, N(C₁₋₄alkyl)₂;

R^{y2} is selected from H, F, Cl, Br, C₁₋₄alkyl, OH, OC₁₋₄alkyl, NH₂, NHC₁₋₄alkyl, N(C₁₋₄alkyl)₂;

R^{y3} is selected from H, F, Cl, Br, C₁₋₄alkyl, OH, OC₁₋₄alkyl, NH₂, NHC₁₋₄alkyl, N(C₁₋₄alkyl)₂;

R^{y4} is selected from H, F, Cl, Br, C₁₋₄alkyl, OH, OC₁₋₄alkyl, NH₂, NHC₁₋₄alkyl, N(C₁₋₄alkyl)₂;

R^{y5} is selected from H, F, CI, Br, C₁₋₄alkyl, OH, OC₁₋₄alkyl, NH₂, NHC₁₋₄alkyl, N(C₁₋₄alkyl)₂;

wherein any two of R^{y1}, R^{y2}, R^{y3}, R^{y4}, and R^{y5} may together form a ring.

In some implementations R^{5a*} is methyl, ethyl, isopropyl, n-propyl, n-butyl, isobutyl, cyclopropyl, cyclobutyl, cyclopentyl, cyclohexyl, CH₂cyclopropyl, CH₂cyclobutyl, CH₂cyclopentyl, CH₂cyclohexyl, CH₂phenyl (i.e., benzyl).

In some implementations, R^{y1}, R^{y2}, R^{y3}, R^{y4}, and R^{y5} are each H.

In other implementations, R^{y2}, R^{y3}, R^{y4}, and R^{y5} are each H, and R^{y1} is F, Cl, Br, C₁₋₄alkyl, OH, OC₁₋₄alkyl, NH₂, NHC₁₋₄alkyl, or N (C₁₋₄alkyl)₂, preferably F, Cl, OH, or OCH₃.

In other implementations, R^{y1}, R^{y3}, R^{y4}, and R^{y5} are each H, and R^{y2} is F, Cl, Br, C₁₋₄alkyl, OH, OC₁₋₄alkyl, NH₂, NHC₁₋₄alkyl, or N (C₁₋₄alkyl)₂, preferably F, Cl, OH, or OCH₃.

In other implementations, R^{y1}, R^{y2}, R^{y4}, and R^{y5} are each H, and R^{y3} is F, Cl, Br, C₁₋₄alkyl, OH, OC₁₋₄alkyl, NH₂, NHC₁₋₄alkyl, or N (C₁₋₄alkyl)₂, preferably F, Cl, OH, or OCH₃.

In certain implementations R^{y1} and R^{y2} together form a ring, for example a fused benzo (i.e., a naphthalene), fused furan (i.e., a benzofuran), a fused pyrrole (i.e., indopyrrole), a fused imidazole (i.e., a benzimidazole), or a fused pyridine (i.e., a quinoloine).

In certain implementations R^{y2} and R^{y3} together form a ring, for example a fused benzo (i.e., a naphthalene), fused furan (i.e., a benzofuran), a fused pyrrole (i.e., indopyrrole), a fused imidazole (i.e., a benzimidazole), or a fused pyridine (i.e., a quinoloine).

In certain implementations the compounds has the formula:

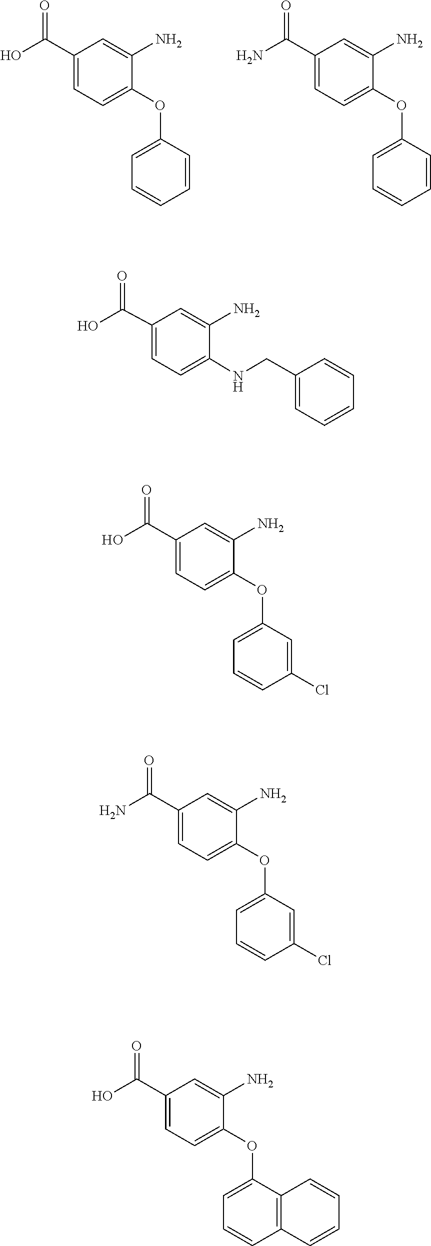 [SMILES: Nc1cc(C(=O)O)ccc1Oc1ccccc1 ; NC(=O)c1ccc(Oc2ccccc2)c(N)c1 ; Nc1cc(C(=O)O)ccc1NCc1ccccc1 ; Nc1cc(C(=O)O)ccc1Oc1cccc(Cl)c1 ; NC(=O)c1ccc(Oc2cccc(Cl)c2)c(N)c1 ; Nc1cc(C(=O)O)ccc1Oc1cccc2ccccc12]

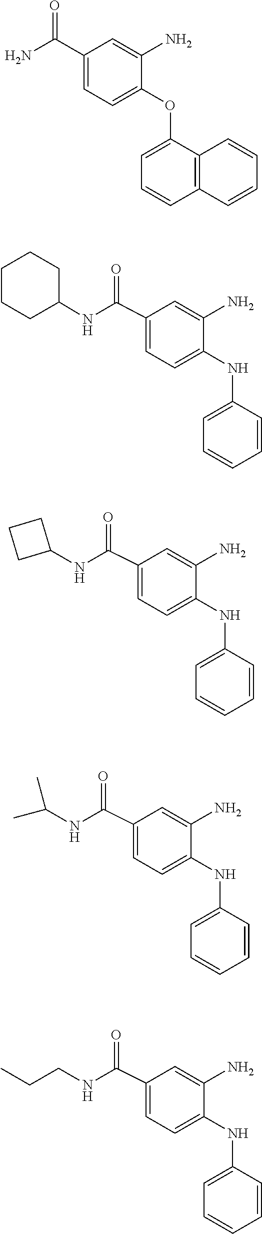 [SMILES: NC(=O)c1ccc(Oc2cccc3ccccc23)c(N)c1 ; Nc1cc(C(=O)NC2CCCCC2)ccc1Nc1ccccc1 ; Nc1cc(C(=O)NC2CCC2)ccc1Nc1ccccc1 ; CC(C)NC(=O)c1ccc(Nc2ccccc2)c(N)c1 ; CCCNC(=O)c1ccc(Nc2ccccc2)c(N)c1]

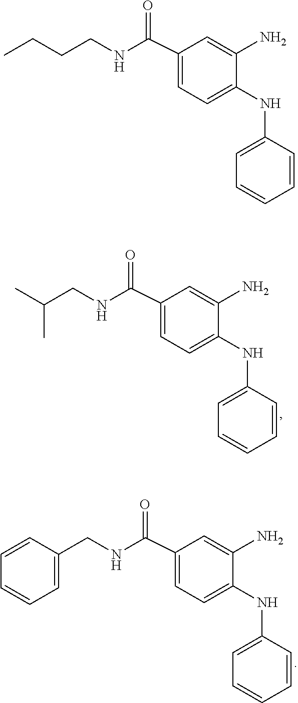 [SMILES: CCCCNC(=O)c1ccc(Nc2ccccc2)c(N)c1 ; CC(C)CNC(=O)c1ccc(Nc2ccccc2)c(N)c1 ; Nc1cc(C(=O)NCc2ccccc2)ccc1Nc1ccccc1]

In certain implementations, the compound is a compound of Formula (3):

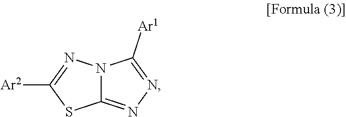

or a pharmaceutically acceptable salt thereof, wherein:

Ar¹ is aryl or C₁₋₈heteroaryl; and

Ar² is aryl or C₁₋₈heteroaryl.

In some implementations Ar¹ is pyridin-2-yl, pyridin-3-yl, or pyridin-4-yl.

The compound of a preceding claim, wherein Ar¹ has the formula:

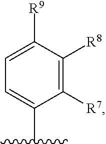

R⁷is F, Cl, Br, I, NO₂, CN, R^{7*}, OR^{7*}, SR^{7*}, N(R^{7*})₂, SO₃R^{7*}, SO₂R^{7*}, SO₂N(R^{7*})₂, C(O)R⁷, C(O)OR^{7*}, OC(O)R^{7*}, C(O)N R^{7*})₂, N(R^{7*})C(O)R^{7*}, OC(O)N(R^{7*})₂, or N(R^{7*})C(O)N(R^{7*})₂, wherein R⁷ is in each case independently selected from hydrogen, C₁₋₈alkyl, aryl, C₁₋₈heteroaryl, C₃₋₈cycloalkyl, or C₁₋₈heterocyclyl; wherein two or more of R⁷ may together form a ring, wherein R⁷ may be substituted one or more times, R^{z1};

R⁸ is F, Cl, Br, I, NO₂, CN, R^{8*}, OR^{8*}, SR^{8*}, N(R^{8*})₂, SO₃R^{8*}, SO₂R^{8*}, SO₂N(R^{8*})₂, C(O)R^{8*}; C(O)OR^{8*}, OC(O)R^{8*}; C(O)N(R^{8*})₂, N(R^{8*})C(O)R^{8*}, OC(O)N(R^{8*})₂, or N(R^{8*})C(O)N(R^{8*})₂, wherein R^{8*} is in each case independently selected from hydrogen, C₁₋₈alkyl, aryl, C₁₋₈heteroaryl, C₃₋₈cycloalkyl, or C₁₋₈heterocyclyl; wherein two or more of R^{8*} may together form a ring, wherein R^{8*} may be substituted one or more times, R^{z1};

R⁹ is F, Cl, Br, I, NO₂, CN, R^{9*}, OR^{9*}, SR^{9*}, N(R^{9*})₂, SO₃R^{9*}, SO₂R^{9*}, SO₂N(R^{9*})₂, C(O)R^{9*}; C(O)OR^{9*}, OC(O)R^{9 *}; C(O)N(R^{9*})₂, N(R^{9*})C(O)R^{9*}, OC(O)N(R^{9*)}₂, or N(R^{9*})C(O)N(R^{9*})₂, wherein R^{9*} is in each case independently selected from hydrogen, C₁₋₈alkyl, aryl, C₁₋₈heteroaryl, C₃₋₈cycloalkyl, or C₁₋₈heterocyclyl; wherein two or more of R^{9*} may together form a ring, wherein R^{9*} may be substituted one or more times, R^{z1};

R^{z1} is in each case independently selected from F, Cl, Br, I, C₁₋₄alkyl, aryl, heteroaryl, OH, OC₁₋₄alkyl, NH₂, NHC₁₋₄alkyl, or N(C₁₋₄alkyl)₂.

In some implementations 2 of R⁷, R⁸, and R⁹ are H, and the other is OH, CH₂NH₂, CH₂OH, C₁₋₆alkyl, F, Cl, Br, or I.

In certain implementations Ar² has the formula:

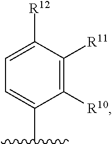

R¹⁰ is F, Cl, Br, I, NO₂, CN, R^{10*}, OR^{10*}, SR^{10*}, N(R^{10*})₂, SO₃R^{10*}, SO₂R^{10*}, SO₂N(R^{10*})₂, C(O)R^{10*}, C(O)OR^{10*}, OC(O)R^{10*}, C(O)N(R^{10*})₂, N(R^{10*})C(O)R^{10*}, OC(O)N(R^{10*})₂, or

N(R^{10*})C(O)N(R^{10*})₂, wherein R^{10*} is in each case independently selected from hydrogen, C₁₋ₛalkyl, aryl, C₁₋₈heteroaryl, C₃₋₈cycloalkyl, or C₁₋₈heterocyclyl; wherein two or more of R^{10*} may together form a ring, wherein R^{10*} may be substituted one or more times, R^{z2};

R¹¹ is F, Cl, Br, I, NO₂, CN, R^{11*}, OR^{11*}, SR^{11*}, N(R^{11*})₂, SO₁₁R^{11*}, SO₂R^{11*}, SO₂N(R^{11*})₂, C(O)R^{11*}, C(O)OR^{11*}, OC(O)R^{11*}, C(O)N(R^{11*})₂, N(R^{11*})C(O)R^{11*}, OC(O)N(R^{11*})₂, or N(R^{11*})C(O)N(R^{11*})₂, wherein R^{11*} is in each case independently selected from hydrogen, C₁₋₁₁alkyl, aryl, C₁₋₁₁heteroaryl, C₃₋₁₁cycloalkyl, or C₁₋₁₁heterocyclyl; wherein two or more of R^{11*} may together form a ring, wherein R^{11*} may be substituted one or more times, R^{z2};

R¹² is F, Cl, Br, I, NO₂, CN, R^{12*}, OR^{12*}, SR^{12*}, N(R^{12*})₂, SO₃R^{12*}, SO₂R^{12*}, SO₂N(R^{12*})₂, C(O)R^{12*}, C(O)OR^{12*}, OC(O)R^{12*}, C(O)N(R^{12*})₂, N(R^{12*})C(O)R^{12*}, OC(O)N(R^{12*})₂, or N(R^{12*})C(O)N(R^{12*})₂, wherein R^{12*} is in each case independently selected from hydrogen, C₁₋₈alkyl, aryl, C₁₋₈heteroaryl, C₃₋₈cycloalkyl, or C₁₋₈heterocyclyl; wherein two or more of R^{12*} may together form a ring, wherein R^{12*} may be substituted one or more times, R^{z2}; and

R^{z2} is in each case independently selected from F, Cl, Br, I, C₁₋₄alkyl, aryl, heteroaryl, OH, OC₁₋₄alkyl, NH₂, NHC₁₋₄alkyl, or N(C₁₋₄alkyl)₂.

In certain implementations, 2 of R¹⁰, R¹¹, and R¹² are H, and the other is OH, CH₂NH₂, CH₂OH, C₁₋₆alkyl, F, Cl, Br, or I.

In certain implementations, the compound is a compound of Formula (3a):

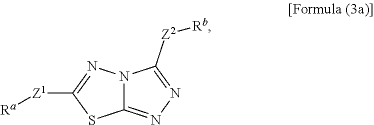

or a pharmaceutically acceptable salt thereof, wherein

Z¹ is selected from null, CH₂, CH₂CH₂, O, NRⁿ¹, S, C(═O), OC(═O), NRⁿ¹(═O),

R^{a} is selected from C₁₋₈alkyl, C₃₋₈cycloalkyl, C₆₋₁₂aryl, C₂₋₁₂heterocyclyl, C₁₋₁₂heteroaryl, wherein

R^{a} may be substituted one or more times by F, Cl, Br, I, C₁₋₄alkyl, aryl, heteroaryl, OH, COOH, CN, NH₂, NHC₁₋₅alkyl, N(C₁₋₅alkyl)₂, or OC₁₋₅alkyl,

wherein R^{a} and Rⁿ¹ may together form a ring; and

Z² is selected from null, CH₂, CH₂CH₂, O, NRⁿ², S, C(═O), OC(═O), NRⁿ²(═O),

R^{b} is selected from C₁₋₈alkyl, OC₁₋₅alkyl, NHC₁₋₅alkyl, N (C₁₋₅alkyl)₂, C₃₋₈cycloalkyl, C₆₋₁₂aryl, C₂₋₁₂heterocyclyl, C₁₋₁₂heteroaryl, wherein Rb may be substituted one or more times by F, Cl, Br, I, C₁₋₄alkyl, aryl, heteroaryl, OH, COOH, CN, NH₂, NHC₁₋₅alkyl, N(C₁₋₅alkyl)₂, or OC₁₋₅alkyl,

wherein R^{b} and Rⁿ² may together form a ring.

In certain implementations, the compound of Formula (3a) can be characterized wherein Z¹ is null, and R^{a} is C₆₋₁₂aryl or C₁₋₁₂heteroaryl. In certain implementations R^{a} is a pyridine ring, a quinoline ring, an isoquinoline ring, a pyrimidine ring, an indole ring, a benzofuran, a benzimidazole ring, a furan ring, a thiophene ring, a pyrrole ring, an oxazole ring, or an imidazole ring. In some implementations, R^{a} is unsubstituted. In other implementations, R^{a} includes one, two, or three substituents, which may be the same or different. In certain implementations R^{a} is substituted one of more times by F, Cl, Br, or OC₁₋₅alkyl. In some implementations R^{a} is a monosubstituted C₆aryl or C₆heteroaryl group. In some implementations R^{a} is substituted with one Cl. In some implementations R^{a} is substituted with one Br. In some implementations R^{a} is substituted with one OC₁₋₅-alkyl group, for example methoxy or ethoxy. In some implementations R^{a} is a disubstituted C₆aryl group. In some implementations R^{a} is substituted with one Cl and one Br. In some implementations R^{a} is substituted with two Cl.

In some implementations, R^{a} has the formula:

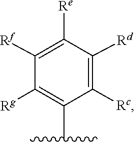

R^{c} is F, Cl, Br, I, NO₂, CN, R^{c*}, OR^{c*}, SR^{c*}, N(R^{c*})₂, SO₃R^{c*}, SO₂R^{c*}, SO₂N(R^{c*})₂, C(O)R^{c*}, C(O)OR^{c*}, OC(O)R^{c*}, C(O)N(R^{c*})₂, N(R^{c*)}C(O)R^{c*}, OC(O)N(R^{c*})₂, or N(R^{c*})C(O)N(R^{c*})₂, wherein R^{c*} is in each case independently selected from hydrogen, C₁₋₈alkyl, aryl, C₁₋₈heteroaryl, C₃₋₈cycloalkyl, or C₁₋₈heterocyclyl; wherein two or more of R^{c*} may together form a ring, wherein R^{c*} may be substituted one or more times by R^{z3};

R^{d} is F, Cl, Br, I, NO₂, CN, R^{d*}, ORª^{*}, SR^{4*}, N(R^{4*})₂, SO₃R^{d*}, SO₂R^{4*}, SO₂N(R^{4*})₂, C(O)R^{d*}, C(O)OR^{d*}, OC(O)R^{d*}; C(O)N(R^{d*})_{2,} N(R^{4*})C(O)R^{d*}, OC(O)N(R^{d*})₂, or N(R^{d*})C(O)N(R^{d*})₂, wherein R^{d*} is in each case independently selected from hydrogen, C₁₋₈alkyl, aryl, C₁₋₈heteroaryl, C₃₋₈cycloalkyl, or C₁₋₈heterocyclyl; wherein two or more of R^{d*} may together form a ring, wherein R^{d*} may be substituted one or more times by R^{z3};

R^{e} is F, Cl, Br, I, NO₂, CN, R^{e*}, OR^{e*}, SR^{e*}, N(R^{e*})₂, SO₃R^{e*}, SO₂R^{e*}, SO₂N(Re*)₂, C(O)R^{e*}, C(O)OR^{e*}, OC(O)R^{e*}, C(O)N(R^{e*})₂, N(R^{e*})C(O)R^{e*}, OC(O)N(R^{e*})₂, or N(R^{e*})C(O)N(R^{e*})₂, wherein R^{e*} is in each case independently selected from hydrogen, C₁₋₈alkyl, aryl, C₁₋₈heteroaryl, C₃₋₈cycloalkyl, or C₁₋₈heterocyclyl; wherein two or more of R^{e*} may together form a ring, wherein R^{e*} may be substituted one or more times by R^{z3}.

R^{f} is F, Cl, Br, I, NO₂, CN, R^{f*}, OR^{f*}, SR^{f*}, N(R^{f*})₂, SO₃R^{f*}, SO₂R^{f*}, SO₂N(R^{f*})₂, C(O)R^{f *}; C(O)OR^{f*}, OC(O)R^{f*}, C(O)N(R^{f*})₂, N(R^{f*})C(O)R^{f*}, OC(O)N(R^{f*})₂, or N(R^{f*})C(O)N(R^{f*})₂, wherein R^{f*} is in each case independently selected from hydrogen, C₁₋₈alkyl, aryl, C₁₋₈heteroaryl, C₃₋₈cycloalkyl, or C₁₋₈heterocyclyl; wherein two or more of R^{f*} may together form a ring, wherein R^{f*} may be substituted one or more times by R^{z3}; and

R^{z3} is in each case independently selected from F, Cl, Br, I, C₁₄alkyl, aryl, heteroaryl, OH, OC₁₋₄alkyl, NH₂, NHC₁₋₄alkyl, or N(C₁₄alkyl)₂.

In some implementations R^{c}, R^{d}, R^{e}, R^{f}, and R^{&} are H, and R^{e} is OH, CH₂NH₂, CH₂OH, C₁₋₆alkyl, OC₁₋₆alkyl, F, Cl, Br, or I. In certain implementations R^{c} is Cl, Br, or OC₁₋₆alkyl.

In some implementations R^{c}, R^{e}, R^{f}, and R^{&} are H, and R^{d} is OH, CH₂NH₂, CH₂OH, C₁₋₆alkyl, OC₁₋₆alkyl, F, Cl, Br, or I. In certain implementations R^{d} is Cl, Br, or OC₁₋₆alkyl. In some implementations R^{e}, R^{d}, R^{f}, and R^{&} are H, and R^{e} is OH, CH₂NH₂, CH₂OH, C₁₋₆alkyl, OC₁₋₆alkyl, F, Cl, Br, or I. In certain implementations R^{e} is Cl, Br, or OC₁₋₆alkyl. In some implementations R^{c}, R^{d}, R^{e}, and R^{&} are H, and R^{f}is OH, CH₂NH₂, CH₂OH, C₁₋₆alkyl, OC₁₋₆alkyl, F, Cl, Br, or I. In certain implementations R^{f}is Cl, Br, or OC₁₋₆alkyl.

In some implementations R^{c}, R^{d}, R^{e}, and Rf are H, and R^{&} is OH, CH₂NH₂, CH₂OH, C₁₋₆alkyl, OC₁₋₆alkyl, F, Cl, Br, or I. In certain implementations R^{e} is Cl, Br, or OC₁₋₆alkyl.

In some implementations R^{c}, R^{d}, R^{e}, and R^{&} are H, and R^{e} and R^{f}are independently selected from OH, CH₂NH₂, CH₂OH, C₁₋₆alkyl, OC₁₋₆alkyl, F, Cl, Br, and I. In certain implementations R^{c} and R^{f} are independently chosen from Cl, Br, or OC₁₋₆alkyl. In certain implementations R^{e} and R^{f} are both Cl.

In certain implementations, the compound of Formula (3a) can be characterized wherein Z²is CH₂ or CH₂CH₂, and R^{b} is C₂₋₁₂heterocyclyl. In certain implementations R^{b} is monocyclic C₄₋₈heterocyclyl group. In certain implementations R^{b} is bicyclic C₆₋₁₀heterocyclyl group. Exemplary bicyclic groups include quinuclidines, 7-azacicyclo [2.2.1] heptanes, 7-azacicyclo [2.1.1] hexanes, tropanes, and the like. In certain implementations R^{b} is C₂₋₁₂heterocyclyl containing one or two nitrogen atoms.

In certain implementations, Z² and R^{b} together form a group having the formula:

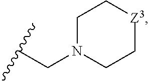

wherein Z³ is selected from null, CH₂, CH₂CH₂, O, S, or NCH₃.

In some implementations, Z² and R^{b} together form a group having the formula:

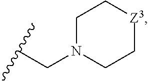

wherein Z³ is selected from null, CH₂, CH₂CH₂, O, S, or NCH₃,

Z¹ is null; and

R^{a} is a C₆aryl group having the formula:

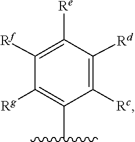

wherein

R^{c} is selected from H, F, Cl, Br, OH, OC₁₋₄alkyl, NH₂, NHC₁₋₄alkyl, N(C₁₋₄alkyl)₂, preferably H, OC₁₋₄alkyl, Cl, or Br;

R^{d} is selected from H, F, Cl, Br, OH, OC₁₋₄alkyl, NH₂, NHC₁₋₄alkyl, N(C₁₋₄alkyl)₂, preferably H, OC₁₋₄alkyl, Cl, or Br;

R^{e} is selected from H, F, Cl, Br, OH, OC₁₋₄alkyl, NH₂, NHC₁₋₄alkyl, N(C₁₋₄alkyl)₂, preferably H, OC₁₋₄alkyl, Cl, or Br;

R^{f} is selected from H, F, CI, Br, OH, OC₁₋₄alkyl, NH₂, NHC₁₋₄alkyl, N(C₁₋₄alkyl)₂, preferably H, OC₁₋₄alkyl, Cl, or Br; and

R^{g} is selected from H, F, Cl, Br, OH, OC₁₋₄alkyl, NH₂, NHC₁₋₄alkyl, N(C₁₋₄alkyl)₂, preferably H, OC₁₋₄alkyl, Cl, or Br;

In certain implementations R^{e} is F, Cl, Br, OH, OC₁₋₄alkyl, NH₂, NHC₁₋₄alkyl, or N(C₁₋₄alkyl)₂, and each of R^{d}, R^{e}, R^{f}, and R^{g} are H

In certain implementations Re is F, Cl, Br, OH, OC₁₋₄alkyl, NH₂, NHC₁₋₄alkyl, or N(C₁₋₄alkyl)₂, R^{f} is F, Cl, Br, OH, OC₁₋₄alkyl, NH₂, NHC₁₋₄alkyl, or N(C₁₋₄alkyl)₂, and each of R^{d}, R^{e}, and R^{g} are H.

In some implementations, the compound has the formula:

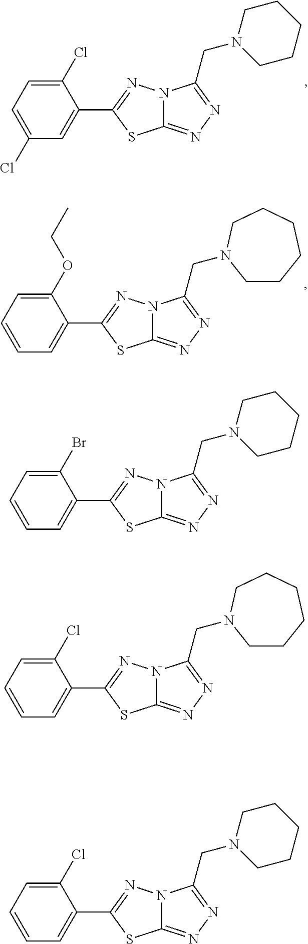 [SMILES: Clc1ccc(Cl)c(-c2nn3c(CN4CCCCC4)nnc3s2)c1 ; CCOc1ccccc1-c1nn2c(CN3CCCCCC3)nnc2s1 ; Brc1ccccc1-c1nn2c(CN3CCCCC3)nnc2s1 ; Clc1ccccc1-c1nn2c(CN3CCCCCC3)nnc2s1 ; Clc1ccccc1-c1nn2c(CN3CCCCC3)nnc2s1]

In some implementations, the compound is a compound of Formula (4):

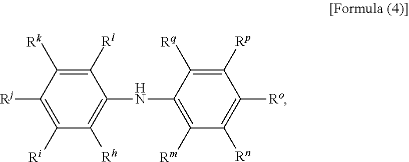

or a pharmaceutically acceptable salt thereof, wherein:

R^{h} is selected from F, Cl, Br, I, NO₂, CN, R^{h*}, OR^{h*}, SR^{h*}, N (R^{h*})₂, SO₃R^{h*}, SO₂R^{h*}, SO₂N(R^{h*})₂, C(O)R^{h*}, C(O)OR^{h*}, OC(O)R^{h*}, C(O)N(R^{h*})₂, N(R^{h*})C(O)R^{h*}, OC(O)N(R^{h*}) ₂, N(R^{h*})C(O)N(R^{h*})₂, or N(R^{h*})C(S)N(R^{h*})₂, wherein R^{h*} is in each case independently selected from hydrogen, C₁₋₈alkyl, aryl, C₁₋₈heteroaryl, C₃₋₈cycloalkyl, or C₁₋₈heterocyclyl; wherein two or more of R^{h*} may together form a ring, wherein R^{h*} may be substituted one or more times by R^{z4}.

Rⁱ is selected from F, Cl, Br, I, NO₂, CN, R^{i*}, OR^{i*}, SR^{i*}, N(R^{i*})₂, SO₃R^{i*}, SO₂R^{i*}, SO₂N(R^{i*})₂, C(O)R^{i*}, C(O)OR^{i*}, OC(O)R^{i*}, C(O)N(R^{i*})₂, N(R^{i*})C(O)R^{i*}, OC(O)N(R^{i*})₂, N(R^{i*})C(O)N(R^{i*})₂, or N(R^{i*})C(S)N(R^{i*})₂, wherein R^{i*} is in each case independently selected from hydrogen, C₁₋₈alkyl, aryl, C₁₋₈heteroaryl, C₃₋₈cycloalkyl, or C₁₋₈heterocyclyl; wherein two or more of R^{i*}may together form a ring, wherein R^{i*} may be substituted one or more times by R^{z4}.

R^{j} is selected from F, Cl, Br, I, NO₂, CN, R^{j*}, OR^{j}, SR^{j*}, N(R^{j*})₂, SO₃R^{j*}, SO₂R^{j*}, SO₂N(R^{j*})₂, C(O)R^{j*}, C(O)OR^{j*}, OC(O)R^{j*}, C(O)N(R^{j*})₂, N(R^{j*})C(O)R^{j*}, OC(O)N(R^{j*})₂, N(R^{j*})C(O)N(R^{j*})₂, or N(R^{j*})C(S)N(R^{j*})₂, wherein R^{j*} is in each case independently selected from hydrogen, C₁₋₈alkyl, aryl, C₁₋₈heteroaryl, C₃₋₈cycloalkyl, or C₁₋₈heterocyclyl; wherein two or more of R^{j*} may together form a ring, wherein R^{j*} may be substituted one or more times by R^{z4}.

R^{k} is selected from F, Cl, Br, I, NO₂, CN, R^{k*}, OR^{k*}, SR^{k*}, N(R^{k*})₂, SO₃R^{k*}, SO₂R^{k*}, SO₂N(R^{k*})₂, C(O)R^{k*}, C(O)OR^{k*}, OC(O)R^{k*}, C(O)N(R^{k*})₂, N(R^{k*})C(O)R^{k*}, OC(O)N(R^{k*})₂, N(R^{k*})C(O)N(R^{k*})₂, or N(R^{k*})C(S)N(R^{k*})₂, wherein R^{k*} is in each case independently selected from hydrogen, C₁₋₈alkyl, aryl, C₁₋₈heteroaryl, C₃₋₈cycloalkyl, or C₁₋₈heterocyclyl; wherein two or more of R^{k*} may together form a ring, wherein R^{k*}may be substituted one or more times by R^{z4}.

R^{l} is selected from F, Cl, Br, I, NO₂, CN, R^{l*}, OR^{l*}, SR^{l*}, N(R^{l*})₂, SO₃R^{l*}, SO₂R^{l*}, SO₂N(R^{l*})₂, C(O)R^{l*}, C(O)OR^{l*}, OC(O)R^{l*}, C(O)N(R^{l*})₂, N(R^{l*})C(O)R^{l*}, OC(O)N(R^{l*})₂, N(R^{l*})C(O)N(R^{l*})₂, or N(R^{l*})C(S)N(R^{l*})₂, wherein R^{l*} is in each case independently selected from hydrogen, C₁₋₈alkyl, aryl, C₁₋₈heteroaryl, C₃₋₈cycloalkyl, or C₁₋₈heterocyclyl; wherein two or more of R^{l*} may together form a ring, wherein R^{l*} may be substituted one or more times by R^{z4}.

R^{m} is selected from F, Cl, Br, I, NO₂, CN, R^{m*}, OR^{m*}, SR^{m*}, N(R^{m*})₂, SO₃R^{m*}, SO₂R^{m*}, SO₂N(R^{m*})₂, C(O)R^{m*}, C(O)OR^{m*}, OC(O)R^{m*}, C(O)N(R^{m*})₂, N(R^{m*})C(O)R^{m*}, OC(O)N(R^{m*})₂, N(R^{m*})C(O)N(R^{m*})₂, or N(R^{m*})C(S)N(R^{m*})₂, wherein R^{m*}is in each case independently selected from hydrogen, C₁₋₈alkyl, aryl, C₁₋₈heteroaryl, C₃₋₈cycloalkyl, or C₁₋₈heterocyclyl; wherein two or more of R^{h*} may together form a ring, wherein R^{m*}may be substituted one or more times by R^{z4}.

Rⁿ is selected from F, Cl, Br, I, NO₂, CN, R^{n*}, OR^{n*}, SR^{n*}, N(R^{n*})₂, SO₃R^{n*}, SO₂R^{n*}, SON(R^{n*})₂, C(O)R^{n*}, C(O)OR^{n*}, OC(O)R^{n*}, C(O)N(R^{n*})₂, N(R^{n*})C(O)R^{n*}, OC(O)N(R^{n*})₂, N(R^{n*})C(O)N(R^{n*})₂, or N(R^{n*})C(S)N(R^{n*})₂, wherein R^{n*}is in each case independently selected from hydrogen, C₁₋₈alkyl, aryl, C₁₋₈heteroaryl, C₁₋₈cycloalkyl, or C₁₋₈heterocyclyl; wherein two or more of R^{n*}may together form a ring, wherein R^{n*}may be substituted one or more times by Rz*.

R^{o} is selected from F, Cl, Br, I, NO₂, CN, R^{o*}, OR^{o*}, SR^{o*}, N(R^{o*})₂, SO₃R^{o*}, SO₂R^{o*}, SO₂N(R^{o*})₂, C(O)R^{o*}, C(O)OR^{o*}, OC(O)R^{o*}, C(O)N(R^{o*})₂, N(R^{o*})C(O)R^{o*}, OC(O)N(R^{o*})₂, N(R^{o*})C(O)N(R^{o*})₂, or N(R^{o*})C(S)N(R^{o*})₂, wherein R^{o*}is in each case independently selected from hydrogen, C₁₋₈alkyl, aryl, C₁₋₈heteroaryl, C₃₋₈cycloalkyl, or C₁₋₈heterocyclyl; wherein two or more of R^{o*}may together form a ring, wherein R^{o*}may be substituted one or more times by R^{z4}.

RP is selected from F, Cl, Br, I, NO₂, CN, R^{p*}, OR^{p*}, SR^{p*}, N(R^{p*})₂, SO₃R^{p*}, SO₂R^{p*}, SO₂N(R^{p*})₂, C(O)R^{p*}, C(O)OR^{p*}, OC(O)R^{p*}, C(O)N(R^{p*})₂, N(R^{p*})C(O)R^{p*}, OC(O)N(R^{p*})₂, N(R^{p*})C(O)N(R^{p*})₂, or N(R^{p*})C(S)N(R^{p*})₂, wherein R^{p*}is in each case independently selected from hydrogen, C₁₋₈alkyl, aryl, C₁₋₈heteroaryl, C₃₋₈cycloalkyl, or C₁₋₈heterocyclyl; wherein two or more of R^{p*}may together form a ring, wherein R^{p*}may be substituted one or more times by R^{z4}.

R^{q} is selected from F, Cl, Br, I, NO₂, CN, R^{q*}, OR^{q*}, SR^{q*}, N(R^{q*})₂, SO₃R^{q*}, SO₂R^{q*}, SO₂N(R^{q*})₂, C(O)R^{q*}, C(O)OR^{q*}, OC(O)R^{q*}, C(O)N(R^{q*})₂, N(R^{q*})C(O)R^{q*}, OC(O)N(R^{q*})₂, N(R^{q*})C(O)N(R^{q*})₂, or N(R^{q*})C(S)N(R^{q*})₂, wherein R^{q*}is in each case independently selected from hydrogen, C₁₋₈alkyl, aryl, C₁₋₈heteroaryl, C₃₋₈cycloalkyl, or C₁₋₈heterocyclyl; wherein two or more of R^{q*}may together form a ring, wherein R^{q*} may be substituted one or more times by R^{z4}; and

R^{z4} is in each case independently selected from F, Cl, Br, I, C₁₋₄alkyl, aryl, heteroaryl, OH, OC₁₋₄alkyl, NH₂, NHC₁₋₄alkyl, or N(C₁₋₄alkyl)₂.

In certain implementations, R^{m}, Rⁿ, R^{o}, R^{p}, and R^{q} are each H.

In certain implementations, R^{m}, Rⁿ, R^{p}, and R^{q} are each H, and R^{o} is selected from F, Cl, Br, C₁₋₄alkyl, OH, OC₁₋₄alkyl, NH₂, NHC₁₋₄alkyl, or N(C₁₋₄alkyl)₂.

In certain implementations, R^{h} is C(O)R^{h*}, C(O)OR^{h*}, OC(O)R^{h*}, C(O)N(R^{h*})₂, N(R^{h*})C(O)R^{h*}, OC(O)N(R^{h*})₂, N(R^{h*})C(O)N(R^{h*})₂, or N(R^{h*})C(S)N(R^{h*})₂, and each of Rⁱ, R^{j}, R^{k}, and R^{l} are selected from H, F, Cl, Br, C₁₋₄alkyl, and OC₁₋₄alkyl. In certain implementations each of Rⁱ, R^{j}, R^{k}, and R^{l} are H.

In certain implementations, Rⁱ is C(O)R^{i*}, C(O)OR^{i*}, OC(O)R^{i*}, C(O)N(R^{i*})₂, N(R^{i*})C(O)R^{i*}, OC(O)N(R^{i*})₂, N(R^{i*})C(O)N(R^{i*})₂, or N(R^{i*})C(S)N(R^{i*})₂, and each of R^{h}, Rⁱ, R^{k}, and R^{l} are selected from H, F, Cl, Br, C₁₋₄alkyl, and OC₁₋₄alkyl. In certain implementations each of R^{h}, Rⁱ, R^{k}, and R^{l} are H.

In certain implementations, R^{j} is C(O)R^{j*}, C(O)OR^{j*}, OC(O)R^{j*}, C(O)N(R^{j*})₂, N(R^{j*})C(O)R^{j*}, OC(O)N(R^{j*})₂, N(R^{j*})C(O)N(R^{j*})₂, or N(R^{j*})C(S)N(R^{j*})₂, and each of R^{h}, Rⁱ, R^{k}, and R^{l} are selected from H, F, Cl, Br, C₁₋₄alkyl, and OC₁₋₄alkyl. In certain implementations, each of R^{h}, Rⁱ, R^{k}, and R^{l} are H.

In certain implementations R^{h} is C(O)R^{h*}, C(O)NHR^{h*}, NHC(O)R^{h*}, OC(O)NHR^{h*}, NHC(O)NHR^{h*}, or NHC(S)NHR^{h*}, and R^{h*} is aryl or C₁₋₈heteroaryl. In certain implementations R^{h} is C(O)NHR^{h*} or NHC(S)NHR^{h*}. Exemplary R^{h*} aryl and C₁₋₈heteroaryl groups include phenyl, naphthyl, pyridinyl, benzofuranyl, indolinyl, benzoimidazyl, and the like. In certain implementations R^{h*} is unsubstituted aryl or unsubstituted C₁₋₈heteroaryl. In certain implementations R^{h*} is substituted aryl or substituted C₁₋₈heteroaryl. In certain implementations R^{h*} is aryl or C₁₋₈heteroaryl, substituted one or more times by F, Cl, Br, C₁₋₄alkyl, OC₁₋₄alkyl, and OC(═O)C₁₋₄alkyl.

In certain implementations Rⁱ is C(O)R^{i*}, C(O)NHR^{i*}, NHC(O)R^{i*}, OC(O)NHR^{i*}, NHC(O)NHR^{i*}, or NHC(S)NHR^{i*}, and R^{i*} is aryl or C₁₋₈heteroaryl. In certain implementations R^{h} is C(O)NHR^{i*} or NHC(S)NHR^{i*}. Exemplary R^{i*} aryl and C₁₋₈heteroaryl groups include phenyl, naphthyl, pyridinyl, benzofuranyl, indolinyl, benzoimidazyl, and the like. In certain implementations R^{i*} is unsubstituted aryl or unsubstituted C₁₋₈heteroaryl. In certain implementations R^{i*} is substituted aryl or substituted C₁₋₈heteroaryl. In certain implementations R^{i*} is aryl or C₁₋₈heteroaryl, substituted one or more times by F, Cl, Br, C₁₋₄alkyl, OC₁₋₄alkyl, and OC(═O)C₁₋₄alkyl.

In certain implementations R^{j} is C(O)R^{j*}, C(O)NHR^{j*}, NHC(O)R^{j*}, OC(O)NHR^{j*}, NHC(O)NHR^{j*}, or NHC(S)NHR^{i*}, and R^{j*} is aryl or C₁₋₈heteroaryl. In certain implementations R^{h} is C(O)NHR^{j*}or NHC(S)NHR^{i*}. Exemplary R^{j*} aryl and C₁₋₈heteroaryl groups include phenyl, naphthyl, pyridinyl, benzofuranyl, indolinyl, benzoimidazyl, and the like. In certain implementations R^{j*}is unsubstituted aryl or unsubstituted C₁₋₈heteroaryl. In certain implementations R^{j*}is substituted aryl or substituted C₁₋₈heteroaryl. In certain implementations R^{i*} is aryl or C₁₋₈heteroaryl, substituted one or more times by F, Cl, Br, C₁₋₄alkyl, OC₁₋₄alkyl, and OC(═O)C₁₋₄alkyl.

Compounds that have inhibitory activity against Salmonella include

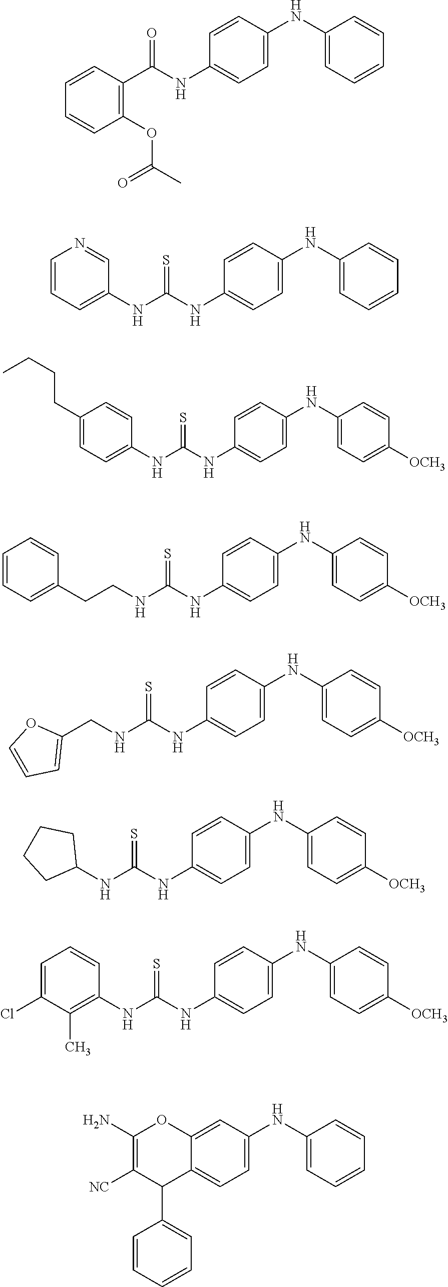

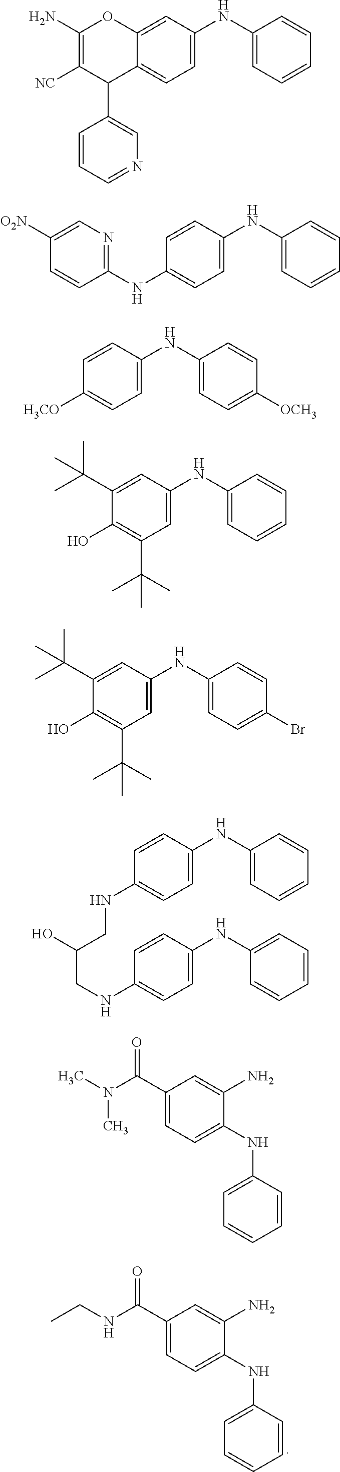

Also disclosed herein are pharmaceutically acceptable compositions including one or more of the compounds disclosed herein. In some implementations the compositions are formulated for oral administration. In some implementations the compositions are formulated for parenteral administration, e.g., intravenous administration, intramuscular administration, or subcutaneous administration. In some implementations, the composition may be formulated as a suppository.

In certain implementations the compositions are formulated as a solid dosage form for oral administration. Such dosage forms, including but not limited to capsules and tablets, can include at least one solid excipient.

In some implementations, the compositions are formulated for parenteral administration. In some implementations, the compositions are sterile aqueous solutions, for instance water for injection, saline, or Ringer's solution, which may be lactated. The aqueous solutions will be formulated at a pH suitable for injections, for example at a pH from 5-8, from 6-8, or from 6.5-7.5. The solutions may include additional excipients such as buffers, antioxidants, and preservatives as known in the art.

Also disclosed herein are methods of treating an infection in a patient in need thereof by administering to the patient a compound disclosed herein. In certain implementations, the infection is a Salmonella infection.

In some implementations the compounds may be administered in combination with one or more additional therapeutic agents. In some implementations the compounds are administered in combination with one or more other antibiotic compounds or probiotics. Exemplary antibiotics that may be administered include β-lactams, cephalosporins, quinolones, and the like. In certain implementations the compound is administered in combination with a Salmonella probiotic that is engineered to compete with Salmonella for nutrients other than F-Asn. In certain implementations the Salmonella probiotic is an engineered Salmonella strain having at least the following mutations: ΔSPI1, ΔSPI2, ΔfraRBDAE4, and ΔasnB80::kan.

### EXAMPLES

The following examples are for the purpose of illustration of the invention only and are not intended to limit the scope of the present invention in any manner whatsoever.

### Example 1—Inhibition of Salmonella

Strains and media: All strains used in this study (Table 1) are derivatives of Salmonella enterica serovar Typhimurium strain ATCC 14028 (hereafter referred to as 14028 or Salmonella).

All strains were routinely grown in LB Broth, Miller (Fisher BioReagents), with shaking at 37° C. High-throughput screening (HTS) was conducted in M9 minimal medium containing 5 mM glucose and 1 mM fructose-asparagine (F-Asn).

| Strain | Genotype | Construction or reference |
|---|---|---|
| 14028 | wild-type Salmonella enterica | American Type Culture Collection |
| | serovar Typhimurium | |
| ASD200 | 14028 ΔSPI1 ΔSPI2 | Microbiol Spectr. 2023; 11(2): e0460622; |
| | | Infect Immun. 2016; 84(7): 2131-40 |
| ASD201 | 14028 ΔSPI1 ΔSPI2 | Microbiol Spectr. 2023; 11(2): e0460622; |
| | Δ(fraR-fraBDAE) | Infect Immun. 2016; 84(7): 2131-40 |
| EFB077 | 14028 ΔSPI1 ΔSPI2 | ASD200 transduced with phage P22 grown |
| | ΔtolC1::kan | on the ΔtolC1::kan mutant strain from |
| | | the mutant collection of Porwollik, |
| | | PLoS One. 2014; 9(7): e99820 |
| EFB078 | ΔSPI1 ΔSPI2 ΔtolC1::kan | ASD201 transduced with phage P22 grown |
| | Δ(fraR-fraBDAE) | on the ΔtolC1::kan mutant strain from |
| | | the mutant collection of Porwollik, |
| | | PLoS One. 2014; 9(7): e99820 |

For safety reasons, all four strains have deletions of the pathogenicity islands, SPIL and SPI2. These mutations render the strains avirulent and have no effect on fraB-dependent toxicity. Except for the deletions of SPI1 and SPI2, ASD200 is otherwise wild-type. ASD201 has an additional deletion of the fra genomic island (fraR-fraBDAF). During growth on F-Asn, inhibitors of FraB should inhibit the growth of ASD200 but have no effect on ASD201. Any inhibitory effects of compounds on ASD201 must be off-target. EFB077 and EFB078 are identical to ASD200 and ASD201, respectively, except that they have an additional mutation in tolC. TolC is an outer membrane protein that participates in efflux of compounds from the cell. These strains allow the determination of compound susceptibility to efflux.

P22 transduction: Bacteriophage P22HTint was grown on a tolC::kan mutant strain from the mutant collection of Porwollik,. The resulting phage lysate was used to infect strains ASD200 and ASD201. Transductants containing the tolC::kan mutation were selected on LB agar plates containing 10 mM EGTA and 50 mg/mL kanamycin. The transductants were struck to isolation twice on LB EGTA kanamycin plates and then tested for pseudolysogeny and phage P22 sensitivity on EBU plates. An isolate of ASD200 transduced with tolC::kan was named EFB077 and an isolate of ASD201 transduced with tolC::kan was named EFB078.

Compounds with fraB-dependent inhibition of Salmonella growth were identified using, four bacterial strains. Overnight cultures of each strain were grown from a single colony inoculated into lysogeny broth (LB) shaking at 37° C. The next morning, each was centrifuged at 18,000×g for 1 min and resuspended in an equal volume of sterile water. Each strain was then diluted 1:100 into sterile water before being added to separate containers of screening medium at a second 1:100 dilution for a final dilution of bacteria of 1:10,000 into screening medium. Each individual strain in screening medium was then dispensed at 30 mI. per well into 384-well plates (Corning 3701) using a BioTek MultiFlo FX Multimode Dispenser with a standard tube dispensing manifold (Agilent Technologies, Inc.). Compounds were added to each well using an Echo 650 Series Acoustic Liquid Handler (Beckman-Coulter), using either 300 nL or 30 nL of compound to achieve a final concentration of 100 mM or 10 mM, respectively. Controls were included on all screening plates. Sixteen wells were used for positive controls which consisted of chloramphenicol dissolved in DMSO at a final chloramphenicol concentration of 50 mg/mL and a final DMSO concentration of 1% (v/v). Sixteen wells were used for negative controls which consisted of DMSO at 1% (v/v). Eight wells were used for a 3-fold dilution series of K19 ranging from 1 mM to 460 nM, and four of these dilution series were included on each plate (32 wells total).

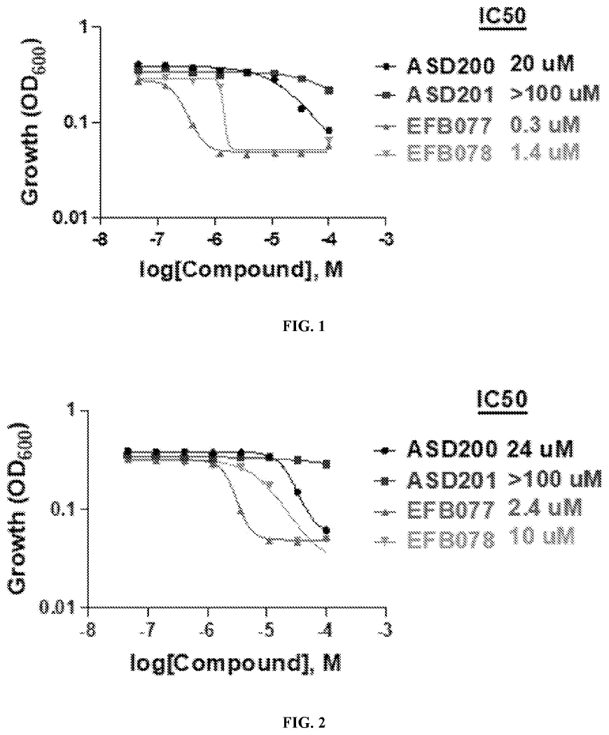

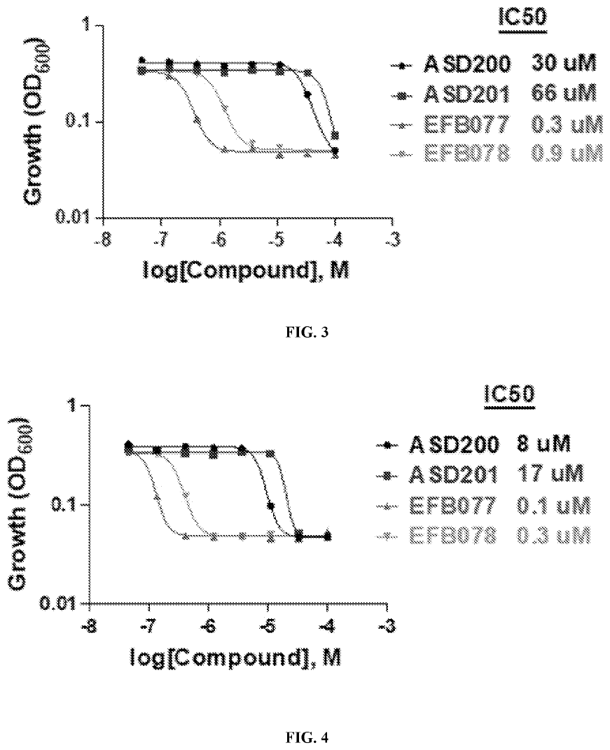

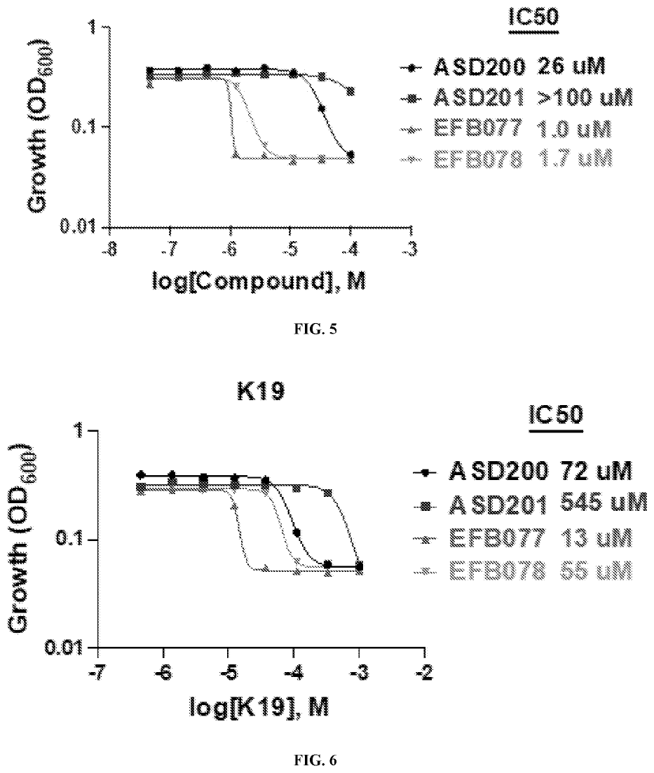

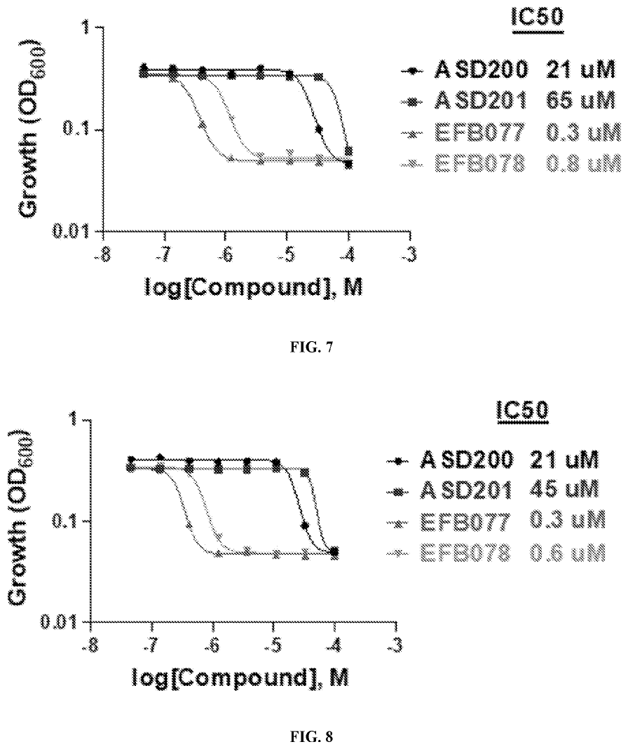

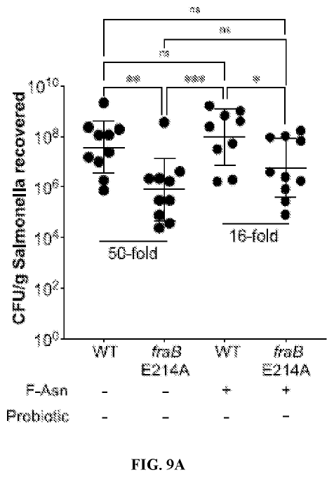

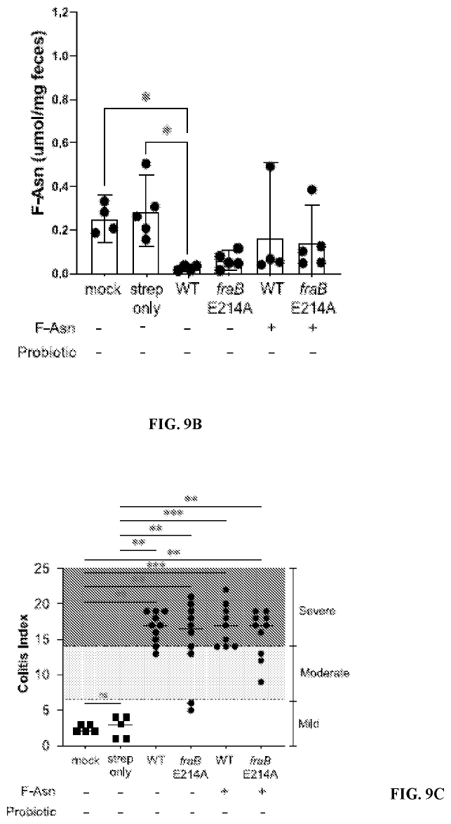

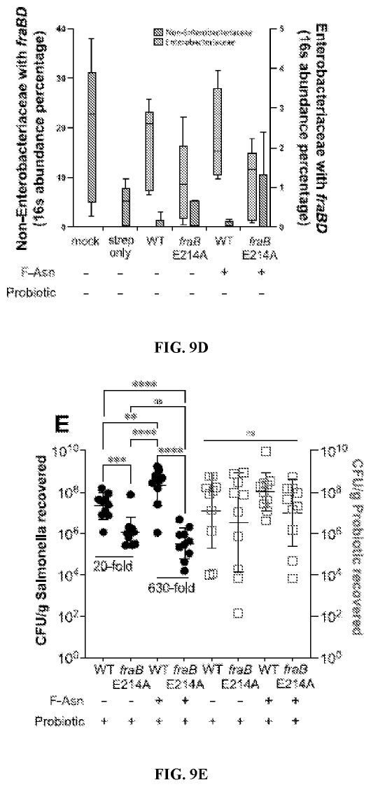

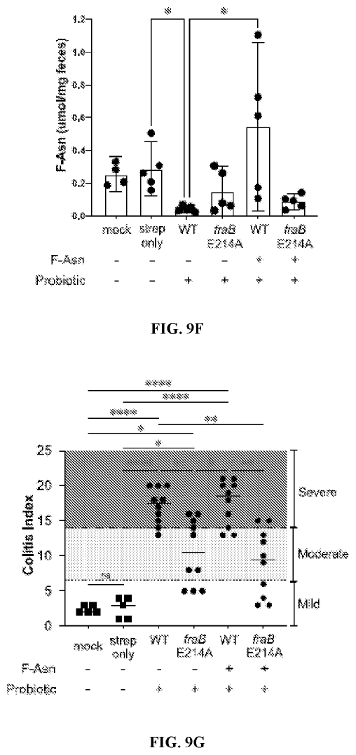

The absorbance of these plates at 600 nm was analyzed with a Tecan Spark Cyto 600 multi-mode plate reader using automated stackers. The screening dataset was aggregated as one csv file and imported into TIBCO Sportfire lead discovery software to generate Z′ factors, signal to noise ratios (S/N), dose responses of the control compound (K19), and scatter plots of each compound using 4 different strains of bacteria. Compounds disclosed herein were validated with IC50 values ranging from 16.4 μM to 30.4 μM. FIGS. 1-8.

### Example 2: A Salmonella Mutant With Probiotic Effect

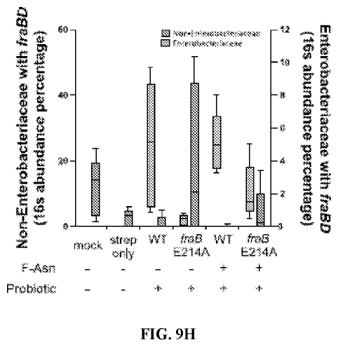

A quadruple mutant designated ASD203 (ASPII ASPI2 AfraRBDAE4 AasnB80:kan) was evaluated for use as a probiotic in combination with FraB inhibitors. This is a strain of Salmonella lacking SPI1 and SPI2 so that it cannot induce inflammation. Additionally, this strain cannot consume F-Asn due to a deletion of the entire fra island (fraR-fraBDAE). The ansB gene was also deleted as this asparaginase is capable of converting F-Asn to F-Asp. The addition of this probiotic should provide an inflammation resistant competitor to Salmonella for all nutrients except F-Asn, effectively forcing the use of F-Asn as a nutrient. The probiotic strain was added in a 1:1 ratio with either wild-type or fraβ mutant Salmonella, in the presence or absence of exogenous F-Asn. The numbers of fraβ mutant bacteria recovered were not significantly different, regardless of the presence of probiotic or F-Asn, however the resulting inflammation was dramatically reduced by the probiotic. FIG. 9A-9H.

Bacterial culture media—All bacterial cultures were maintained in LB broth (Fisher BioReagents) or on agar plates with 1.5% (wt/vol) agar (Fisher BioReagents). Strains used in this study are listed in Table 1. Antibiotics were added to media for plasmid maintenance or marker selection at the following concentrations: kanamycin (kan, 50 μg/ml), chloramphenicol (cam, 30 μg/ml), ampicillin (amp, 100 μg/ml). EGTA was used to stop P22 phage infection at 10 mM. Screening for pseudolysogens after P22 transductions used Evans Blue-Uranine (EBU) plates consisting of (per L): 10 g tryptone, 5 g yeat extract, 5 g NaCl, 1.5 g glucose, 1.5% agar. After autoclaving, media was supplemented with 40 mL of 12.5% K₂HPO₄, 1.25 mL 1% Evans Blue solution, 2.5 mL 1% Uranine 32.

Minimum inhibitory concentration—The MIC for each strain was calculated from a growth assay after 15-hour incubation at 37° C. using three biological replicates with three technical replicates each (nine total data points). IC₅₀ and IC₉₀ were calculated with Prism (GraphPad).

P22 transductions—Strain ASD5900 was constructed by transducing the fraB1::cam marker from strain MA5900 into 14028 using phage P22HTint. To do this, phage P22HTint was propagated on MA5900 and the resulting phage were used to infect 14028. Transductants were selected on LB cam supplemented with 10 mM EGTA. Colonies were streaked to isolation twice on LB cam supplemented with 10 mM EGTA. Transductants were tested for pseudolysogeny and full length LPS by streaking against P22HTint on EBU plates.

fraD qRT-PCR—Strains were grown in LB in a roller drum at 37° C. overnight. The cultures were then washed twice and resuspended in sterile water. The washed cells were used to inoculate 50 mL of M9 minimal medium supplemented with 5 mM glucose resulting in a 1:100 dilution. These cultures were allowed to grow for four hours to mid-exponential phase at which point F-Asn was added to 1 mM and incubation continued for 30 minutes, at which point 15 mL aliquots were taken for qRT-PCR and for intracellular 6-P-F-Asp measurements, and were harvested by spinning at 5,000×g for 10 minutes at 4° C. Pellets were stored at −20° C. From the qRT-PCR aliquots, RNA was extracted using the Purelink RNA Miniprep kit (Invitrogen). Briefly, pellets were thawed on ice and resuspended in freshly made lysis buffer and lysed cells run through the column per the manufacturer's protocol. After the RNA was eluted from the column in 50 μL RNase free water, the RNA was treated for DNA contamination using the Turbo DNA-free kit (Invitrogen) by adding 10× DNase buffer and 2 U of DNase and incubated for 30 min at 37° C. After incubation, 5 μL of DNase inactivation reagent was added to the reaction, incubated for 5 minutes at room temperature with periodic mixing and the spun at 19,000×g for 1 min. The supernatant was removed and placed in an RNase-free microcentrifuge tube and stored at −20° C. For reverse transcription (RT), 100 ng of total RNA was used for cDNA synthesis using Superscript VILO (Invitrogen) per the manufacturer's protocol. For quantitation of fraD expression, 1 μL of cDNA was used in a qPCR reaction using iTaq SYBR Green Supermix (BioRad) with either 300 μM of primers BA3405 and BA3406 (fraD) or BA2745 and BA2746 (rpoB) in the CFX96 Touch Real-time PCR machine (BioRad). Amplification efficiencies for each primer pair (fraD BA3405 and BA3506 was 91.48%; rpoB BA2745 and BA2746 was 90.42%; see Table 1) were calculated using a 10-fold dilution series standard curve of pooled cDNA from all samples from one of the biological replicates. The Ct values were analyzed using the Pfaffl method where the rpoB control was used as the internal calibrator and the wild-type fral) expression is the reference condition and all mutant fraD expression is relative to the wild-type.

Measurement of 6-P-F-Asp concentration in fraB mutants using mass spectrometry: To measure intracellular 6-P-F-Asp, 15 ml samples of cells were collected and pelleted (see qRT-PCR section above) for each biological replicate. Three biological replicates were collected on separate occasions. Each cell pellet was roughly 100 μL. 200 μL of LC grade water was added to give 300 μL for each biological replicate. This was split into three aliquots, each 100 μL. Two aliquots were used as analytical replicates for each analysis. 6-P-F-Asp was extracted from each 100 μL aliquot (equivalent to 5 mL of cells), by adding 250 μL of chilled LC grade water and 500 μL of chilled methanol (Fisher Optima LC/MS grade, Fisher Scientific) with 20 nmol [¹³C]-F-Asn added as internal standard. Aliquots from wild-type Salmonella cells were spiked with 0, 20, 80, 160, 200, 400 or 600 nmol of 6-P-F-Asp to generate a standard curve. The cell suspension was then vortexed for 2 minutes to facilitate pellet disruption and metabolite extraction. Samples were then subjected to ten cycles of ultrasonication (30 s each with 30 s intervals in between) using a Bioruptor® Pico (Diagenode). Following cell lysis, samples were centrifuged at 16,000×g for 15 min at 4° C. and the supernatants were transferred to new tubes and dried under vacuum (SpeedVac Concentrator, Thermo Scientific). Before mass spectrometry analysis, these dried pellets were resuspended in 50 μL water: acetonitrile, 98%:2% with 0.1% (v/v) formic acid (LC-MS grade, Thermo Scientific) and filtered using a 0.2 μm PTFE filter (Thermo Scientific). The supernatant of the flow-through fraction was then injected for liquid chromatographic-mass spectrometric analysis. 10 μL of each sample (equivalent to 1 ml of cells) was introduced into an Ultimate 3000 Ultra Performance LC (Thermo Scientific) system with an HSS T3, C18 column (Waters, 2.1 μm×100 mm, 1.8 μm) and coupled into a triple quadrupole mass spectrometer (Thermo Quantiva TQ-S). Mobile phases were buffer A, 0.1% (v/v) formic acid (LC-MS grade, Thermo Scientific) in water and buffer B, 0.1% (v/v) formic acid in acetonitrile. A gradient separation started with 2% B for 3 min at a flow rate of 100 μl/min and then followed by gradient: 3-4 min, 2-10% B; 4-7 min, 10-98% B; 7-7.5 min, 98% B; 7.5-7.6 min, 98-2% B; 7.6-10 min, 2% B. The mass spectrometer was operated in positive ion electrospray ionization mode (ESI+) with a capillary voltage at 4 kV, source temperature 100° C., desolvation temperature 350° C., sheath gas flow 12 L/min and auxiliary gas flow 13 L/min. The gas flow rate for the collision cell was 0.15ml/min. A multiple reaction monitoring (MRM) mode was used. While transitions m/z 376→242 of 6-P-F-Asp with collision energy 15 eV was selected for quantitation, m/z 301→283 of [13C]-F-Asn with collision energy 13eV was used for normalization. Skyline (v20.2, MacCoss Lab, Department of Genome Sciences, University of Washington, Seattle, WA, USA) was used for calculating the peak area of transition.

Mass Spec of F-Asn from mouse fecal samples—For sample preparation, 1 mL of the extraction solvent (Water/Acetonitrile, 80/20, %/%) with 13C6-F-Asn was added to the fecal sample for F-ASN extraction. The fecal slurry was lysed by the bath sonication with Bioruptor® (Diagenode, Belgium) followed by centrifugation for protein precipitation. The supernatant was transferred and dried. The dried residual was reconstituted and injected into LC-MS/MS. For the standard curve analysis, different amounts of F-Asn standard compound (5.15, 12.9, 25.8, 51.5, 128.8, 257.7, 515.5, 1288.6 nmol) were spiked into the extraction solvent and were prepared as described above. F-Asn and ¹³C₆-F-Asn were separated using Dionex Ultimate™ 3000 RSLC liquid chromatography system (Thermo Scientific, USA) with a C18 column (Kinetex® 1.7 μm C18 100 Å, LC Column 100×2.1 mm, Phenomenex Inc, USA)). The mobile phase comprised 0.1% aqueous formic acid solution for mobile phase A and 0.1% formic acid in acetonitrile for mobile phase B. Gradient elution was applied at a flow rate of 100 μL/min. 2% mobile phase B was held for 3 min, ramped to 98% B over 7 min, and then kept until 7.5 min. Subsequently, re-equilibration for 2.5 min at 2% B was performed, thereby giving an overall runtime of 10 min. Thermo Quantiva triple quadrupole mass spectrometer (Thermo Scientific, USA) was used in a positive-ion mode.

Mouse modeling experiments—For competition experiments, a 1:1 mixture of two different bacterial strains were inoculated intragastrically (i.g.) and then recovered from ceca to determine the competitive index (CI) of the two strains. Streptomycin-treated mice were administered 20 mg of streptomycin 24 hours prior to inoculation with Salmonella strains. Streptomycin-treated C57BL/6, Swiss Webster, and streptomycin-treated CBA/J mice were inoculated with 10⁷ total CFU of Salmonella. CBA/J mice that were not treated with streptomycin were inoculated with 10⁹ total CFU. CI was determined by plating on media selective for WT (LB cam) and mutant (LB kan). For the competitions with the fraB F214A mutant which is not marked, CI was determined by screening 52 individual colonies recovered from XLD plates for antibiotic resistance by patching on LB plates containing the appropriate antibiotic, thus the limit of detection on these experiments is less than two logs. For the single infection mouse modeling, streptomycin-treated Swiss-Webster mice were inoculated with a single bacterial strain at 10⁷ total CFU. When indicated, 735 mg of F-Asn was administered intragastrically (i.g.) daily, which is 25 mM in 100 μL. When indicated, a probiotic strain was administered in a 1:1 mixture with the tested Salmonella strain. Ceca and feces were harvested on day four post-infection. CFU enumeration was determined by plating on media selective for WT (LB cam) and the mutant (LB kan). For the fraB E214A mutant, which is not marked, CFU enumeration was determined by plating on XLD.

Limiting dilution series—Cecal contents from a 1:1 competition of WT and fraB80::kan mutant were used to calculate the rate of spontaneous mutants resistant to F-Asn intoxication in vivo. The homogenized ceca from ten mice were serially diluted by 10-fold dilutions in microcentrifuge tubes in 1×M9 medium supplemented with 5 mM glucose 5 mM F-Asn and kanamycin as the diluent. This medium is selective for the fraB80::kan mutant. In a 96 well plate 200 μL of each dilution from one mouse was added to the first row of each column. One 96 well plate was used for dilutions of cecum content from one mouse. From the first row, each dilution was further diluted 10-fold down the column in 1×M9 medium supplemented with 5 mM glucose 5 mM F-Asn and kanamycin. All plates were lidded and incubated at 37° C. overnight. After incubation the lids were removed and the absorbance was read at 600 nm in the Spectra Max M2 (Molecular Devices). Based on the total CFU/mL obtained from plating the homogenized ceca, we were able to calculate the total CFU added to each well and the mutation rate frequency.

Mutation fluctuation tests—To calculate the mutation fluctuation of F-Asn resistance, 94colonies of fraB80::kan were inoculate into the wells of a 96 well plate containing 1×M9 medium supplemented with 5 mM glucose and 5 mM F-Asn. One well was inoculated with wild type as a positive growth control and one well was inoculated with wild type but the growth medium was supplemented with chloramphenicol as a negative growth control. Three of the wells were immediately titered by serial dilution and drip plated into LB plates to enumerating the starting number of CFU in each well. The plate was lidded and incubated at 37° C. for 16 hours. After incubation, three of the wells were titered by serial dilution and drip plated on LB plates to enumerate the total CFU in each well after incubation. Then, the lid was removed and the absorbance was read at 600 nm in a Spectra Max M2 plate reader (Molecular Devices). The mutation frequency was calculated as mutation rate (m)=−ln(#of wells with no growth/total number of wells). Of the 91 total wells, 30 wells had no resistant mutations, therefore the frequency was calculated as 1.1 mutation events per culture. The rate of spontaneous mutation was calculated by assuming one colony on an LB agar plate started with one cell. Since individual colonies were picked by toothpick and placed into the 96 well plate we assume the total CFU of a colony equals the number of cell divisions from one cell seeding that single colony and as three of the wells were immediately titered for enumeration, this CFU number is assumed to be the number of CFU in the colony that was initially picked from the agar plate. The mean of all three wells enumerated was 1.7e7 CFU/well, therefore the spontaneous mutation rate was calculated as 1.7e7/1.1=6.5×10⁻⁸ mutants per cell division.

Isolation of transposon mutants resistant to F-Asn-A mutant library was created by transducing a T-POP transposon (Tn10dTet [del25]) into the fraB80::kan mutant background, HMB206. To prepare the lysate, Salmonella enterica serovar Typhimurium strain TH3923 was grown overnight in LB at 30°° C. The cells were washed and subcultured to OD550 of 0.39. 1 mL of 0.5 mg/mL mitomycin C was added to induce phage production, and the cells were allowed to lyse with shaking at 37° C. for 4 hours. The solution was filter sterilized, transferred to a glass tube, and 100 μL of chloroform was added. The transduction was performed immediately after lysate preparation. A 5 mL culture of HMB206 carrying a plasmid, pNK2880, that encodes a Tn10 transposase with relaxed target specificity, was grown in LB with ampicillin at 37° C. overnight with shaking. In a sterile flask, 5 mL of the TH3923 lysate was added to 5 mL of the HMB206 +pNK2880 culture, and incubated for 25 minutes at 37° C. The cells were washed twice in LB with EGTA and resuspended in 50 mL of LB with EGTA for a 60 minute outgrowth. At this point, the number of mutants in the culture was determined by titering on LB with tetracycline. Tetracycline was then added to the remainder of the culture and allowed to grow at 37° C. overnight at which point glycerol stocks were created and stored at −80° C. The library contained 25,000 mutants.

A selection was applied to the library to obtain mutants that could grow in the presence of F-Asn. Ten culture tubes with 5 mL of minimal media with 5 mM glucose, 5 mM F-Asn, kanamycin, and tetracycline were each inoculated with 100 μL of library and grown at 37° C. overnight. The overnight selections were titered and 100 μL of each dilution was plated on LB with tetracycline and EGTA. One colony was selected from each of the 10 cultures and struck to isolation twice on LB with tetracycline and EGTA. Another 96 mutants were obtained using a 96-well format in which 2 μL of library was inoculated into 200 μl. of medium and one mutant was isolated from each well after overnight growth at 37° C. Growth of all mutants in M9+glucose+F-Asn was confirmed. To identify the transposon insertion point, inverse PCR was performed on genomic DNA that was purified using the GenElute bacterial genomic DNA kit (Sigma). The genomic DNA was first digested with the restriction enzyme NlaIII (NEB). The reaction was incubated at 37° C. for 3 hours, and the enzyme was heat inactivated at 65° C. for 20 minutes. A dilute ligation was performed with T4 DNA ligase at 15° C. overnight (NEB). The ligation was purified with a QIAprep® Spin Miniprep Kit (Qiagen). The DNA was then digested with DraI for 3 hours at 37° C. and heat inactivated for 20 minutes at 65° C. (NEB). PCR was performed with primers IPCRF and IPCRR³⁹. The PCR product was purified with QIAquick® PCR Purification Kit (Qiagen) and submitted for Sanger sequencing to the Plant-Microbe Genomics Facility at Ohio State University.

Histopathology—Samples of the proximal colon were removed from mice, and a portion was immersion fixed in 10% neutral buffered formalin, processed by routine methods, and embedded in paraffin wax by the Comparative Pathology and Mouse Phenotyping Shared Resource (CPMPSR) at the Ohio State University. Sections (4 μm) were stained with hematoxylin and eosin (H&E) and scored in a blinded fashion by a veterinary pathologist, board certified by the American College of Veterinary Pathologists (ACVP) as previously described.

16S rRNA gene amplicon sequencing and analysis—Total nucleic acids were extracted using the Quick-DNA Fecal/Soil Microbe Microprep Kit (Zymo Research) and stored at −20° C. until sequencing. DNA was submitted for amplicon sequencing at Argonne National Lab at the Next Generation Sequencing facility using Illumina MiSeq with 2×251 bp paired end reads following established HMP protocols. Briefly, universal primers 515F and 806R were used for PCR amplification of the V4 hypervariable region of 16S rRNA gene using 30 cycles. The 515F primer contained a unique sequence tag to barcode each sample. Both primers contained sequencer adapter regions. The sequencing data was then processed using QIIME2. Amplicon Sequence Variants (ASVs) were determined using DADA2 and taxonomy was predicted based on a naive Bayes classifier built from 16S sequences in SILVA. To predict which 16S sequences were potential non-Enterobacteriaceae F-Asn consumers, 16S sequences were taken from the sequences used in and clustered with sequences from 16S sequencing at 99% identity using VSEARCH. Then based on taxonomic assignment of the 16S sequences the summed relative abundance of non-Enterobacteriaceae ASVs which clustered with sequences from was reported across all experimental conditions.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-69. (canceled)

70. A method for treating an infection in a subject in need thereof, comprising administering to the subject a compound having of Formula (1), Formula (2), Formula (3), or Formula (4): 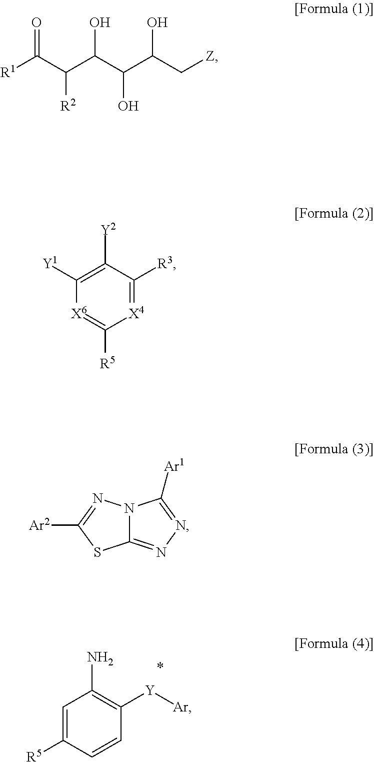
or a pharmaceutically acceptable salt thereof, wherein:
R¹ is OR^{1a} or N(R^{1a})₂, wherein R^{1a} is in each case independently selected from H, C₁₋₈alkyl, aryl, C₁₋₈heteroaryl, C₃₋₈cycloalkyl, or C₁₋₈heterocyclyl;
R² is H or OH;
Z is Z¹—X, wherein Z¹ is null, NH, S, O, CH₂, CH₂O, OCH₂, OCH₂O, CH₂NH, NHCH₂, CH₂S, SCH₂, X is P(O)(OR^{p})₂, SO₂N(R^{p})₂, SO₂R^{p*}, wherein R^{p} is in each case independently selected from H or C₁₋₈alkyl, and R^{p*} is C₁₋₈alkyl;
one of Y¹ and Y² is NH₂, and the other is Y*—Ar, wherein Y* is null, NH, or O, and Ar is aryl or C₁₋₈heteroaryl;
X⁴ is N or CR⁴;
X⁶ is N or CR⁶;
R³ is F, Cl, Br, I, NO₂, CN, R^{3a*}, OR^{3a*}, SR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*}is in each case independently selected from hydrogen, C₁₋₈alkyl, aryl, C₁₋₈heteroaryl, C₃₋₈cycloalkyl, or C₁₋₈heterocyclyl; wherein two or more of R^{3a} may together form a ring;
R⁴ is F, Cl, Br, I, NO₂, CN, R^{4a*}, OR^{4a*}, SR^{4a*}, N(R^{4a*})₂, SO₄R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a*}is in each case independently selected from hydrogen, C₁₋₈alkyl, aryl, C₁₋₈heteroaryl, C₃₋₈cycloalkyl, or C₁₋₈heterocyclyl; wherein two or more of R^{4a} may together form a ring;
R⁵ is F, Cl, Br, I, NO₂, CN, R^{5a*}, OR^{5a*}, SR^{5a*}, N(R^{5a*})₂, SO₅R^{5a*}, SO₂R^{5a*}, SO₂N(R^{5a*})₂, C(O)R^{5a*}; C(O)OR^{5a*}, OC(O)R^{5a*}; C(O)N(R^{5a*})₂, N(R^{5a*})C(O)R^{5a*}, OC(O)N(R^{5a*})₂, N(R^{5a*})C(O)N(R^{5a*})₂, wherein R^{5a*}is in each case independently selected from hydrogen, C₁₋₈alkyl, aryl, C₁₋₈heteroaryl, C₃₋₈cycloalkyl, or C₁₋₈heterocyclyl; wherein two or more of R^{5a} may together form a ring;
R⁶ is F, Cl, Br, I, NO₂, CN, R^{6a*}, OR^{6a*}, SR^{6a*}, N(R^{6a*})₂, SO₆R^{6a*}, SO₂R^{6a*}, SO₂N(R^{6a*})₂, C(O)R^{6a*}; C(O)OR^{6a*}, OC(O)R^{6a*}; C(O)N(R^{6a*})₂, N(R^{6a*})C(O)R^{6a*}, OC(O)N(R^{6a*})₂, N(R^{6a*})C(O)N(R^{6a*})₂, wherein R^{6a*}is in each case independently selected from hydrogen, C₁₋₈alkyl, aryl, C₁₋₈heteroaryl, C₃₋₈cycloalkyl, or C₁₋₈heterocyclyl; wherein two or more of R^{6a} may together form a ring;
Ar¹ is aryl or C₁₋₈heteroaryl;
Ar² is aryl or C₁₋₈heteroaryl;
Ar is C₆aryl C₁₋₈heteroaryl, C₃₋₈cycloalkyl, or C₁₋₈heterocyclyl; and
R⁵ is C(O)OR^{5*} or C(O)N(R^{5*})₂, preferably C(O)N(R^{5*})₂, wherein R^{5*} is independently selected from H, C₁₋₈alkyl, and C₃₋₈cycloalkyl.

71. The method of claim 70, comprising administering the compound of Formula (1), wherein R¹ is OC₁₋₈alkyl and R² is OH.

72. The method of claim 70, comprising administering the compound of Formula (1), wherein Z¹ is null, O, CH₂, CH₂O, OCH₂, or OCH₂O.

73. The method of claim 70, comprising administering the compound of Formula (1), wherein X is P(O)(OH)₂, SO₂NH₂, SO₂CH₃.

74. The method of claim 70, comprising administering the compound of Formula (2), wherein Y² is NH₂, and X⁴ is C—R⁴, wherein R⁴ is F, Cl, Br, I, CN, R^{4a*}, OR^{4a*}, C(O)OR^{4a*}, or C(O)N(R^{4a*})₂.

75. The method of claim 70, comprising administering the compound of Formula (2), wherein R⁵ is F, Cl, Br, I, R^{4a*}, or OR^{4a*}, wherein R^{4a} is Hor C₁₋₄alkyl.

76. The method of claim 70, comprising administering the compound of Formula (2), wherein Y² is NH₂, and X⁴ is C—R⁴, wherein R₄C(O)N(R^{4a*})₂, and R^{4a} is independently selected from H, C₁₋₄alkyl, or C₁₋₈heterocyclyl.

77. The method of claim 70, comprising administering the compound of Formula (2), wherein Y² is NH₂, and X⁴ is C—R⁴, wherein R⁴C(O)N(R^{4a*})₂, wherein both of R^{4a} together form a ring.

78. The method of claim 70, comprising administering the compound of Formula (3), wherein Ar¹ is pyridin-2-yl, pyridin-3-yl, or pyridin-4-yl, or Ar¹ has the formula: 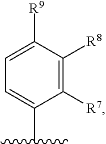
R⁷ is F, Cl, Br, I, NO₂, CN, R^{7a*}, OR^{7a*}, SR^{7a*}, N(R^{7a*})₂, SO₇R^{7a*}, SO₂R^{7a*}, SO₂N(R^{7a*})₂, C(O)R^{7a*}; C(O)OR^{7a*}, OC(O)R^{7a*}; C(O)N(R^{7a*})₂, N(R^{7a*})C(O)R^{7a*}, OC(O)N(R^{7a*})₂, N(R^{7a*})C(O)N(R^{7a*})₂, wherein R^{7a*} is in each case independently selected from hydrogen, C₁₋₈alkyl, aryl, C₁₋₈heteroaryl, C₃₋₈cycloalkyl, or C₁₋₈heterocyclyl; wherein two or more of R^{7a} may together form a ring;
R⁸ is F, Cl, Br, I, NO₂, CN, R^{8a*}, OR^{8a*}, SR^{8a*}, N(R^{8a*})₂, SO₈R^{8a*}, SO₂R^{8a*}, SO₂N(R^{8a*})₂, C(O)R^{8a*}; C(O)OR^{8a*}, OC(O)R^{8a*}; C(O)N(R^{8a*})₂, N(R^{8a*})C(O)R^{8a*}, OC(O)N(R^{8a*})₂, N(R^{8a*})C(O)N(R^{8a*})₂, wherein R^{8a*} is in each case independently selected from hydrogen, C₁₋₈alkyl, aryl, C₁₋₈heteroaryl, C₃₋₈cycloalkyl, or C₁₋₈heterocyclyl; wherein two or more of R^{8a} may together form a ring;
R⁹ is F, Cl, Br, I, NO₂, CN, R^{9a*}, OR^{9a*}, SR^{9a*}, N(R^{9a*})₂, SO₉R^{9a*}, SO₂R^{9a*}, SO₂N(R^{9a*})₂, C(O)R^{9a*}; C(O)OR^{9a*}, OC(O)R^{9a*}; C(O)N(R^{9a*})₂, N(R^{9a*})C(O)R^{9a*}, OC(O)N(R^{9a*})₂, N(R^{9a*})C(O)N(R^{9a*})₂, wherein R^{9a*} is in each case independently selected from hydrogen, C₁₋₈alkyl, aryl, C₁₋₈heteroaryl, C₃₋₈cycloalkyl, or C₁₋₈heterocyclyl; wherein two or more of R^{9a} may together form a ring.

79. The method of claim 70, comprising administering the compound of Formula (3), wherein Ar¹ has the formula: 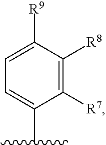
wherein 2 of R⁷, R⁸, and R⁹ are H, and the other is OH, CH₂NH₂, CH₂OH, C₁₋₆alkyl, F, Cl, Br, or I.

80. The method of claim 70, comprising administering the compound of Formula (3), wherein Ar² has the formula: 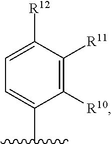
R¹⁰ is F, Cl, Br, I, NO₂, CN, R^{10a*}, OR^{10a*}, SR^{10a*}, N(R^{10a*})₂, SO₁₀R^{10a*}, SO₂R^{10a*}, SO₂N(R^{10a*})₂, C(O)R^{10a*}; C(O)OR^{10a*}, OC(O)R_{10a*}; C(O)N(R^{10a*})₂, N(R^{10a*})C(O)R^{10a*}, OC(O)N(R^{10a*})₂, N(R^{10a*})C(O)N(R^{10a*})₂, wherein R^{10a*} is in each case independently selected from hydrogen, C₁₋₈alkyl, aryl, C₁₋₈heteroaryl, C₃₋₈cycloalkyl, or C₁₋₈heterocyclyl; wherein two or more of R^{10a*} may together form a ring;
R¹¹ is F, CI, Br, I, NO₂, CN, R^{11a*}, OR^{11a*}, SR^{11a*}, N(R^{11a*})₂, SO₁₁R^{11a*}, SO₂R^{11a*}, SO₂N(R^{11a*})₂, C(O)R^{11a*}; C(O)OR^{11a*}, OC(O)R^{11a*}; C(O)N(R^{11a*})₂, N(R^{11a*})C(O)R^{11a*}, OC(O)N(R^{11a*})₂, N(R^{11a*})C(O)N(R^{11a*})₂, wherein R^{11a*}is in each case independently selected from hydrogen, C₁₋₁₁alkyl, aryl, C₁₋₁₁heteroaryl, C₃₋₁₁cycloalkyl, or C₁₋₁₁heterocyclyl; wherein two or more of R^{11a} may together form a ring;
R¹² is F, Cl, Br, I, NO₂, CN, R^{12a*}, OR^{12a*}, SR^{12a*}, N(R^{12a*})₂, SO₁₂R^{12a*}, SO₂R^{12a*}, SO₂N(R^{12a*})₂, C(O)R^{12a*}; C(O)OR^{12a*}, OC(O)R^{12a*}; C(O)N(R^{12a*})₂, N(R^{12a*})C(O)R^{12a*}, OC(O)N(R^{12a*})₂, N(R^{12a*})C(O)N(R^{12a*})₂, wherein R^{12a*}is in each case independently selected from hydrogen, C₁₋₈alkyl, aryl, C₁₋₈heteroaryl, C₃₋₈cycloalkyl, or C₁₋₈heterocyclyl; wherein two or more of R^{12a} may together form a ring.

81. The method of claim 80, wherein 2 of R¹⁰, R¹¹, and R¹² are H, and the other is OH, CH₂NH₂, CH₂OH, C₁₋₆alkyl, F, Cl, Br, or I.

82. The method of claim 70, comprising administering the compound of Formula (4), having the formula: 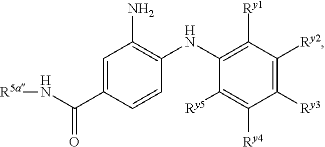
wherein
R^{5a*}is selected from H C₁₋₈alkyl, and C₃₋₈cycloalkyl;
R^{y1} is selected from H, F, Cl, Br, C₁₋₄alkyl, OH, OC₁₋₄alkyl, NH₂, NHC₁₋₄alkyl, N(C₁₋₄alkyl)₂;
R^{y2} is selected from H, F, Cl, Br, C₁₋₄alkyl, OH, OC₁₋₄alkyl, NH₂, NHC₁₋₄alkyl, N(C₁₋₄alkyl)₂;
R^{y3} is selected from H, F, Cl, Br, C₁₋₄alkyl, OH, OC₁₋₄alkyl, NH₂, NHC₁₋₄alkyl, N(C₁₋₄alkyl)₂;
R^{y4} is selected from H, F, Cl, Br, C₁₋₄alkyl, OH, OC₁₋₄alkyl, NH₂, NHC₁₋₄alkyl, N(C₁₋₄alkyl)₂;
R^{y5} is selected from H, F, Cl, Br, C₁₋₄alkyl, OH, OC₁₋₄alkyl, NH₂, NHC₁₋₄alkyl, N(C₁₋₄alkyl)₂;
wherein any two of R^{y1}, R^{y2}, R^{y3}, R^{y4}, and R^{y5} may together form a ring.

83. The method of claim 82, wherein R^{5a*}is methyl, ethyl, isopropyl, n-propyl, n-butyl, isobutyl, cyclopropyl, cyclobutyl, cyclopentyl, cyclohexyl, CH₂cyclopropyl, CH₂cyclobutyl, CH₂cyclopentyl, CH₂cyclohexyl, or CH₂phenyl.

84. The method of claim 82, wherein R^{y2}, R^{y3}, R^{y4}, and R^{y5} are each H, and R^{y1} is F, Cl, Br, C₁₋₄alkyl, OH, OC₁₋₄alkyl, NH₂, NHC₁₋₄alkyl, or N(C₁₋₄alkyl)₂.

85. The method of claim 70, wherein the infection is a Salmonella infection.

86. The method of claim 85, further comprising administering to the patient a Salmonella probiotic that is engineered to compete with Salmonella for nutrients other than F-Asn.

87. The method of claim 85, further comprising administering to the patient an engineered Salmonella strain having at least the following mutations: DSPI1, DSPI2, DfraRBDAE4, and DasnB80::kan.

88. A compound having the formula: 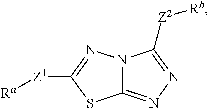
or pharmaceutically acceptable salt thereof, wherein
Z¹ is selected from null, CH₂, CH₂CH₂, O, NRⁿ¹, S, C(═O), OC(═O), NRⁿ¹(═O),
R^{a} is selected from C₁₋₈alkyl, C₃₋₈cycloalkyl, C₆₋₁₂aryl, C₂₋₁₂heterocyclyl, C₁₋₁₂heteroaryl, wherein
R^{a} may be substituted one or more times by F, Cl, Br, I, C₁₋₄alkyl, aryl, heteroaryl, OH, COOH, CN, NH₂, NHC₁₋₈alkyl, N(C₁₋₈alkyl)₂, or OC₁₋₈alkyl,
wherein R^{a} and Rⁿ¹ may together form a ring; and
Z² is selected from null, CH₂, CH₂CH₂, O, NRⁿ², S, C(═O), OC(═O), NRⁿ²(═O),
R^{b} is selected from C₁₋₈alkyl, OC₁₋₅alkyl, NHC₁₋₈alkyl, N(C₁₋₅alkyl)₂, C₃₋₈cycloalkyl, C₆₋₁₂aryl, C₂₋₁₂heterocyclyl, C₁₋₁₂heteroaryl, wherein R^{b} may be substituted one or more times by F, Cl, Br, I, C₁₋₄alkyl, aryl, heteroaryl, OH, COOH, CN, NH₂, NHC₁₋₈alkyl, N(C₁₋₅alkyl)₂, or OC₁₋₈alkyl,
wherein R^{b} and Rⁿ² may together form a ring; or
a compound having the formula: 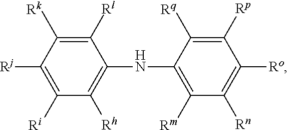
or a pharmaceutically acceptable salt thereof, wherein:
R^{h} is selected from F, Cl, Br, I, NO₂, CN, R^{h*}, OR^{h*}, SR^{h*}, N(R^{h*})₂, SO₃R^{h*}, SO₂R^{h*}, SO₂N(R^{h*})₂, C(O)R^{h*}, C(O)OR^{h*}, OC(O)R^{h*}, C(O)N(R^{h*})₂, N(R^{h*})C(O)R^{h*}, OC(O)N(R^{h*})₂, N(R^{h*})C(O)N(R^{h*})₂, or N(R^{h*})C(S)N(R^{h*})₂, wherein R^{h*} is in each case independently selected from hydrogen, C₁₋₈alkyl, aryl, C₁₋₈heteroaryl, C₃₋₈cycloalkyl, or C₁₋₈heterocyclyl; wherein two or more of R^{h*} may together form a ring, wherein R^{h*} may be substituted one or more times by R^{z4}.
Rⁱ is selected from F, Cl, Br, I, NO₂, CN, R^{i*}, OR^{i*}, SR^{i*}, N(R^{i*})₂, SO₃R^{i*}, SO₂R^{i*}, SO₂N(Ri)₂, C(O)R^{i*}, C(O)OR^{i*}, OC(O)R^{i*}, C(O)N(R^{i*})₂, N(R^{i*})C(O)R^{i*}, OC(O)N(R^{i*})₂, N(R^{i*})C(O)N(R^{i*})₂, or N(R^{i*})C(S)N(R^{i*})₂, wherein R^{i*}is in each case independently selected from hydrogen, C₁₋₈alkyl, aryl, C₁₋₈heteroaryl, C₃₋₈cycloalkyl, or C₁₋₈heterocyclyl; wherein two or more of R^{i*} may together form a ring, wherein R^{i*} may be substituted one or more times by R^{z4}.
Rⁱ is selected from F, Cl, Br, I, NO₂, CN, R^{i*}, OR^{i*}, SR^{i*}, N(R^{i*})₂, SO₃R^{i*}, SO₂R^{i*}, SO₂N(R^{i*})₂, C(O)R^{i*}, C(O)OR^{i*}, OC(O)R^{i*}, C(O)N(R^{i*})₂, N(R^{i*})C(O)R^{i*}, OC(O)N(R^{i*})₂, N(R^{i*})C(O)N(R^{i*})₂, or N(R^{i*})C(S)N(R^{i*})₂, wherein R^{i*} is in each case independently selected from hydrogen, C₁₋₈alkyl, aryl, C₁₋₈heteroaryl, C₃₋₈cycloalkyl, or C₁₋₈heterocyclyl; wherein two or more of R^{i*} may together form a ring, wherein R^{i*} may be substituted one or more times by R^{z4}.
R^{k} is selected from F, Cl, Br, I, NO₂, CN, R^{k*}, OR^{k*}, SR^{k*}, N(R^{k*})₂, SO₃R^{k*}, SO₂R^{k*}, SO₂N(R^{k*})₂, C(O)R^{k*}, C(O)OR^{k*}, OC(O)R^{k*}, C(O)N(R^{k*})₂, N(R^{k*})C(O)R^{k*}, OC(O)N(R^{k*})₂, N(R^{k*})C(O)N(R^{k*})₂, or N(R^{k*})C(S)N(R^{k*})₂, wherein R^{k*} is in each case independently selected from hydrogen, C₁₋₈alkyl, aryl, C₁₋₈heteroaryl, C₃₋₈cycloalkyl, or C₁₋₈heterocyclyl; wherein two or more of R^{k*} may together form a ring, wherein R^{k*} may be substituted one or more times by R^{z4}.
R^{l} is selected from F, Cl, Br, I, NO₂, CN, R^{l*}, OR^{l*}, SR^{l*}, N(R^{l*})₂, SO₃R^{l*}, SO₂R^{l*}, SO₂N(R^{l*})₂, C(O)R^{l*}, C(O)OR^{l*}, OC(O)R^{l*}, C(O)N(R^{l*})₂, N(R^{l*})C(O)R^{l*}, OC(O)N(R^{l*})₂, N(R^{l*})C(O)N(R^{l*})₂, or N(R^{l*})C(S)N(R^{l*})₂, wherein R^{l*} is in each case independently selected from hydrogen, C₁₋₈alkyl, aryl, C₁₋₈heteroaryl, C₃₋₈cycloalkyl, or C₁₋₈heterocyclyl; wherein two or more of R^{l*} may together form a ring, wherein R^{l*} may be substituted one or more times by R^{z4}.
R^{m} is selected from F, Cl, Br, I, NO₂, CN, R^{m*}, OR^{m*}, SR^{m*}, N(R^{m*})₂, SO₃R^{m*}, SO₂R^{m*}, SO₂N(R^{m*})₂, C(O)R^{m*}, C(O)OR^{m*}, OC(O)R^{m*}, C(O)N(R^{m*})₂, N(R^{m*})C(O)R^{m*}, OC(O)N(R^{m*})₂, N(R^{m*})C(O)N(R^{m*})₂, or N(R^{m*})C(S)N(R^{m*})₂, wherein R^{m*} is in each case independently selected from hydrogen, C₁₋₈alkyl, aryl, C₁₋₈heteroaryl, C₃₋₈cycloalkyl, or C₁₋₈heterocyclyl; wherein two or more of R^{h*} may together form a ring, wherein R^{m*} may be substituted one or more times by R_{z4}.
Rⁿ is selected from F, Cl, Br, I, NO₂, CN, R^{n*}, OR^{n*}, SR^{n*}, N(R^{n*})₂, SO₃R^{n*}, SO₂R^{n*}, SO₂N(R^{n*})₂, C(O)R^{n*}, C(O)OR^{n*}, OC(O)R^{n*}, C(O)N(R^{n*})₂, N(R^{n*})C(O)R^{n*}, OC(O)N(R^{n*})₂, N(R^{n*})C(O)N(R^{n*})₂, or N(R^{n*})C(S)N(R^{n*})₂, wherein R^{n*} is in each case independently selected from hydrogen, C₁₋₈alkyl, aryl, C₁₋₈heteroaryl, C₃₋₈cycloalkyl, or C₁₋₈heterocyclyl; wherein two or more of R^{n*} may together form a ring, wherein R^{n*} may be substituted one or more times by R^{z4}.
R^{o} is selected from F, Cl, Br, I, NO₂, CN, R^{o*}, OR^{o*}, SR^{o*}, N(R^{o*})₂, SO₃R^{o*}, SO₂R^{o*}, SO₂N(R^{o*})₂, C(O)R^{o*}, C(O)OR^{o*}, OC(O)R^{o*}, C(O)N(R^{o*})₂, N(R^{o*})C(O)R^{o*}, OC(O)N(R^{o*})₂, N(R^{o*})C(O)N(R^{o*})₂, or N(R^{o*})C(S)N(R^{o*})₂, wherein R^{o*} is in each case independently selected from hydrogen, C₁₋₈alkyl, aryl, C₁₋₈heteroaryl, C₃₋₈cycloalkyl, or C₁₋₈heterocyclyl; wherein two or more of R^{o*} may together form a ring, wherein R^{o*} may be substituted one or more times by R^{z4}.
R^{p} is selected from F, Cl, Br, I, NO₂, CN, R^{p*}, OR^{p*}, SR^{p*}, N(R^{p*})₂, SO₃R^{p*}, SO₂R^{p*}, SO₂N(R^{p*})₂, C(O)R^{p*}, C(O)OR^{p*}, OC(O)R^{p*}, C(O)N(R^{p*})₂, N(R^{p*})C(O)R^{p*}, OC(O)N(R^{p*})₂, N(R^{P*})C(O)N(R^{p*})₂, or N(R^{p*})C(S)N(R^{p*})₂, wherein R^{p*} is in each case independently selected from hydrogen, C₁₋₈alkyl, aryl, C₁₋₈heteroaryl, C₃₋₈cycloalkyl, or C₁₋₈heterocyclyl; wherein two or more of R^{p*} may together form a ring, wherein R^{p*} may be substituted one or more times by R^{z4}.
R^{q} is selected from F, Cl, Br, I, NO₂, CN, R^{q*}, OR^{q*}, SR^{q*}, N(R^{q*})₂, SO₃R^{q*}, SO₂R^{q*}, SO₂N(R^{q*})₂, C(O)R^{q*}, C(O)OR^{q*}, OC(O)R^{q*}, C(O)N(R^{q*})₂, N(R^{q*})C(O)R^{q*}, OC(O)N(R^{q*})₂, N(R^{q*})C(O)N(R^{q*})₂, or N(R^{q*})C(S)N(R^{q*})₂, wherein R^{q*} is in each case independently selected from hydrogen, C₁₋₈alkyl, aryl, C₁₋₈heteroaryl, C₃₋₈cycloalkyl, or C₁₋₈heterocyclyl; wherein two or more of R^{q*} may together form a ring, wherein R^{q*} may be substituted one or more times by R^{z4}; and
R^{z4} is in each case independently selected from F, Cl, Br, I, C₁₋₄alkyl, aryl, heteroaryl, OH, OC₁₋₄alkyl, NH₂, NHC₁₋₄alkyl, or N(C₁₋₄alkyl)₂; or
a compound having the formula: 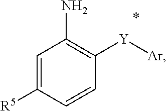
or pharmaceutically acceptable salt thereof, wherein
Ar is C₆aryl C₁₋₈heteroaryl, C₃₋₈cycloalkyl, or C₁₋₈heterocyclyl; and
R⁵ is C(O)OR^{5*} or C(O)N(R^{5*})₂, preferably C(O)N(R^{5*})₂, wherein R^{5*} is independently selected from H, C₁₋₈alkyl, and C₃₋₈cycloalkyl; or
a compound having the formula: 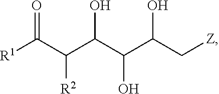
or a pharmaceutically acceptable salt thereof, wherein:
R¹ is OR^{1a} or N(R^{1a})₂, wherein R^{1a} is in each case independently selected from H, C₁₋₈alkyl, aryl, C₁₋₈heteroaryl, C₃₋₈cycloalkyl, or C₁₋₈heterocyclyl;
R² is H or OH;
Z is Z¹—X, wherein Z¹ is null, NH, S, O, CH₂, CH₂O, OCH₂, OCH₂O, CH₂NH, NHCH₂, CH₂S, SCH₂, X is P(O)(OR^{p})₂, SO₂N(R^{p})₂, SO₂R^{p*}, wherein R^{p} is in each case independently selected from H or C₁₋₈alkyl, and R^{p*} is C₁₋₈alkyl; or
a compound having the formula: 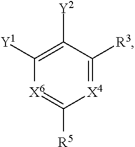
one of Y¹ and Y² is NH₂, and the other is Y*—Ar, wherein Y* is null, NH, or O, and Ar is aryl or C₁₋₈heteroaryl;
X⁴ is N or CR⁴;
X⁶ is N or CR⁶;
R³ is is F, Cl, Br, I, NO₂, CN, R^{3a*}, OR^{3a*}, SR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl; wherein two or more of R^{3a*} may together form a ring;
R⁴ is is F, Cl, Br, I, NO₂, CN, R^{4a*}, OR^{4a*}, SR^{4a*}, N(R^{4a*})₂, SO₄R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a*} is in each case independently selected from hydrogen, C₁₋₈alkyl, aryl, C₁₋₈heteroaryl, C₃₋₈cycloalkyl, or C₁₋₈heterocyclyl; wherein two or more of R^{4a*} may together form a ring;
R⁵ is is F, Cl, Br, I, NO₂, CN, R^{5a*}, OR^{5a*}, SR^{5a*}, N(R^{5a*})₂, SO₅R^{5a*}, SO₂R^{5a*}, SO₂N(R^{5a*})₂, C(O)R^{5a*}; C(O)OR^{5a*}, OC(O)R^{5a*}; C(O)N(R^{5a*})₂, N(R^{5a*})C(O)R^{5a*}, OC(O)N(R^{5a*})₂, N(R^{5a*})C(O)N(R^{5a*})₂, wherein R^{5a*} is in each case independently selected from hydrogen, C₁₋₈alkyl, aryl, C₁₋₈heteroaryl, C₃₋₈cycloalkyl, or C₁₋₈heterocyclyl; wherein two or more of R^{5a*} may together form a ring;
R⁶ is is F, Cl, Br, I, NO₂, CN, R^{6a*}, OR^{6a*}, SR^{6a*}, N(R^{6a*})₂, SO₆R^{6a*}, SO₂R^{6a*}, SO₂N(R^{6a*})₂, C(O)R^{6a*}; C(O)OR^{6a*}, OC(O)R^{6a*}; C(O)N(R^{6a*})₂, N(R^{6a*})C(O)R^{6a*}, OC(O)N(R^{6a*})₂, N(R^{6a*})C(O)N(R^{6a*})₂, wherein R^{6a*} is in each case independently selected from hydrogen, C₁₋₈alkyl, aryl, C₁₋₈heteroaryl, C₃₋₈cycloalkyl, or C₁₋₈heterocyclyl; wherein two or more of R^{6a*} may together form a ring;
Ar¹ is aryl or C₁₋₈heteroaryl; and
Ar² is aryl or C₁₋₈heteroaryl.

89. An engineered Salmonella strain having at least the following mutations: DSPI1, DSPI2, DfraRBDAE4, and DasnB80::kan.
